(19) United States
(12) Patent Application Publication
(10) Pub. No.: US 2022/0259243 A1
(43) Pub. Date: Aug. 18, 2022

(54) IRON(III) AND GALLIUM(III) METAL ORGANIC POLYHEDRA, METHODS OF MAKING SAME, AND USES THEREOF

(71) Applicant: The Research Foundation for The State University of New York, Amherst, NY (US)

(72) Inventors: Janet R. MORROW, Williamsville, NY (US); Timothy R. COOK, Amherst, NY (US); Gregory E. SOKOLOW, Buffalo, NY (US); Amber RIVERA, Buffalo, NY (US)

(21) Appl. No.: 17/622,117

(22) PCT Filed: Jun. 22, 2020
(86) PCT No.: PCT/US20/39025

§ 371 Date: Dec. 22, 2021

Related U.S. Application Data
(60) Provisional application No. 62/865,182, filed on Jun. 22, 2019.

(51) Int. Cl. C07F 15/02; C07F 5/00; A61K 49/10

(52) U.S. Cl. CPC C07F 15/025; A61K 49/101; C07F 5/00

(57) ABSTRACT

Compounds may have at least two structural units, which may be referred to as ligands. Each structural unit includes at least one spacer group and two or more donor groups. Compounds may have two or more iron(III) cations, one or more of which may be a high-spin iron(III) cation or high-spin iron(III) cations, two or more gallium(III) cations, or at least one iron(III) cation, one or more of which may be a high-spin iron(III) cation or high-spin iron(III) cations, and at least one gallium(III) cation, where the iron(III) cation(s) and/or the gallium(III) cation(s) coordinate to the donor groups. The compounds may be self-assembled cages. A composition may include one or more of the compound(s) and a pharmaceutically accepted carrier. Methods of imaging use one or more of the compound(s) and/or one or more of the composition(s).

### CROSS REFERENCE TO RELATED APPLICATIONS

This application claims priority to U.S. Provisional Application No. 62/865,182, filed on Jun. 22, 2019, the disclosure of which is hereby incorporated by reference.

### BACKGROUND OF THE DISCLOSURE

To date, nearly all clinically-used contrast agents contain gadolinium (Gd as trivalent Gd(III)) despite the fact that a substantial proportion of patients in the US population (ca 10%) are considered at risk for being given Gd(III) contrast agents. There are new concerns that Gd(III) based MRI contrast agents are leading to the deposition of Gd(III) into brain, bone and skin of all patients. Alternatives to Gd(III) contrast agents that involve biologically relevant transition metal ions include high-spin Mn(II) and high-spin Fe(III) complexes

Most existing Fe(III) MRI contrast agents contain simple linear chelates including those with an ethylene diamine backbone with a combination of phenol and carboxylate pendents such as, for example, EHBG (N,N′-ethylenebis[(2-hydroxybenzyl)glycine). Another type contains polyaminocarboxylate ligands, such as, for example, Fe(III) complexes of EDTA. A third type contains the bacterial siderophore, desferrioxamine (DFO).

All of these complexes have drawbacks including reduction potentials that are amenable for ROS generation, low aqueous solubility and difficulty of synthetic modification. Also, the aqueous solution chemistry of Fe(III) complexes is dominated by the formation of insoluble complexes with hydroxides and bridging oxide ligands.

### SUMMARY OF THE DISCLOSURE

Compounds of the present disclosure provide an alternative to Gd(III) contrast agents. Compounds of the present disclosure can provide desirable control over spin and oxidation state of the Fe(III) cations in the molecules and/or desirable interactions of the molecules with inner-sphere and outer-sphere water. The compounds may stabilize Fe(III) in high-spin form. Also, the compounds may exhibit desirable aqueous solution chemistry.

In an aspect, the present disclosure provides compounds and ligands. The compounds may be referred to as metal organic polyhedra, supramolecular clusters, complexes, or self-assembled cages. The compounds may be self-assembled iron(III) or gallium(IIII) molecules. In various examples, the compounds or ligands are a salt, a partial salt, a hydrate, a polymorph, or a stereoisomer, or a mixture thereof.

Iron is present in the compounds in its trivalent form (Fe(III)). The Fe(III) may be high-spin Fe(III). Gallium is present in the compounds in its trivalent form (Ga(III)).

A compound may comprise at least two structural units (which may be referred to as ligands). Each structural unit (or a ligand) may comprise at least one spacer group (which may be referred to a linker group) and two or more donor groups (which may be referred to as chelating groups and may have one or two groups that coordinate to an iron(III) cation), and at least two iron(III) cations (which may be high-spin iron(III) cations). A compound may comprise various donor groups. A donor group has one or more functional group(s) that can coordinate to an iron(III) cation or a gallium(III) cation. A compound may comprise various spacer groups. A spacer group has at least two covalent bonds, where each covalent bond is to a donor group.

In an aspect, the present disclosure provides compositions. A composition may comprise one or more compound(s) of the present disclosure. A composition may comprise one or more compound(s) of the present disclosure and a pharmaceutically acceptable carrier. A composition may also comprise one or more protein(s).

In an aspect, the present disclosure provides methods of making self-assembled iron(III) and/or Ga(III) compounds. Illustrative, non-limiting examples of methods of making a structural unit/ligands and compounds of the present disclosure are described in Schemes 1 and 2.

In an aspect, the present disclosure provides uses of compounds of the present disclosure. The compounds may be used in imaging methods. The imaging methods (e.g., magnetic resonance imaging methods and/or positron emission tomography methods, and the like) of the present disclosure can be used to image a cell, tissue, organ, vasculature, or a part thereof. The cell, tissue, organ, vasculature may be a part of an individual.

### BRIEF DESCRIPTION OF THE FIGURES

FIG. 1 (Scheme 1) shows non-limiting examples of chelating groups. Any of the linker groups may be substituted as described herein. An R₁ group may be an alkyl group, an aryl group, a heteroaryl group, a polyether PEG group, or a targeting group.

FIG. 2 (Scheme 2) shows non-limiting examples of linker groups. Any of the linker groups may be substituted as described herein.

FIG. 3 (Scheme 3) shows an example of a ligand and various protonation/coordination states.

FIG. 4 (Scheme 4) shows an example of a general ligand design.

FIG. 5 (Scheme 5) shows non-limiting examples metal-ligand architectures.

FIG. 6 (Scheme 1) shows non-limiting examples of ligands and linking groups. Any of the ligands and linker groups may be substituted as described herein.

FIG. 7 shows non-limiting examples of ligands. Any of the ligands may be substituted as described herein.

FIG. 8 shows non-limiting examples of compounds of the present disclosure. Any of the compounds may be substituted as described herein.

FIG. 9 shows T1 weighted MRI images of mice injected with Fe4A6 at dosages of 50 μmol/kg, 25 μmol/kg, and 12.5 μmol/kg taken at 45 minutes and 4 hours post intravenous injection.

FIG. 10 shows maximum intensity projections of mice injected with Fe4A6 at dosages of 50 μmol/kg, 25 μmol/kg, and 12.5 μmol/kg taken at 45 minutes and 4 hours post intravenous injection.

FIG. 11 shows T1 weighted MRI images of mouse injected with a dose of 12.5 μmol/kg Fe4A6. Post-injection image shows significant cardiac enhancement.

FIG. 12 shows measurement of ΔT1 rate (s)⁻¹ over a 4 hour period post-injection at dosages of 50, 25, and 12.5 μmol/kg of Fe4A6.

FIG. 13 shows measurement of ΔT1 rate (s)⁻¹ over a 45 minute period post-injection at dosages of 50, 25, and 12.5 μmol/kg of Fe4A6. Contrast enhancement appears to be proportional to dose with the exception of 50 μmol/kg and 25 μmol/kg Fe4A6 in the kidney which shows signs of saturation.

FIG. 14 show an electronic spectrum of a K₅(NEt₄)₇[Fe₄A₆] complex in phosphate buffered saline solution at pH 7.4, 37° C. The UV-vis spectrum of the complex was monitored over 12 hours to show no change (all of the individual minute curves overlap).

FIG. 15 shows the absorbance at 499 nm of a K₅(NEt₄)₇[Fe₄A₆] complex in phosphate buffered saline solution at pH 7.4, 37° C. was monitored over 10 hours. No change in the absorbance was observed for different concentrations of complex, with and without human serum albumin (HSA).

FIG. 16 shows ORTEP of Me₄B, C₂₄H₂₄N₂O₆, orthorhombic, Fdd2 space group.

FIG. 17 shows ORTEP of Me₄C, C₂₄H₂₄N₂O₆, triclinic, P-1 space group.

FIG. 18 shows ORTEP of Me₄D, C₂₄H₂₄N₂O₆, monoclinic, P2₁/c space group.

FIG. 19 shows ORTEP of Me₄E, C₂₄H₃₀N₂O₆, monoclinic, P2₁/n space group.

FIG. 20 shows ORTEP of Fe₂C₂, C₄₈H₄₂Fe₂K₂N₅O_{20.5}, monoclinic, 12 space group. Counter-ions omitted.

FIG. 21 shows sample FT-ICRMS isotopic pattern for the fragment K(NEt₄)₂[Fe₂C₂(μ-MeO)₂]. Experimental (top) compared to simulated pattern (bottom).

FIG. 22 shows a scheme describing the synthesis of HOPO-based ligands.

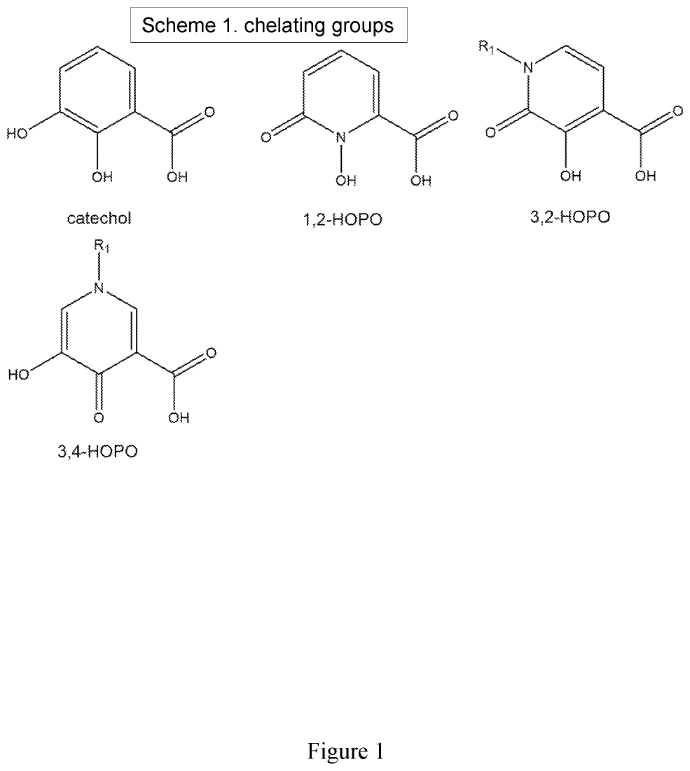

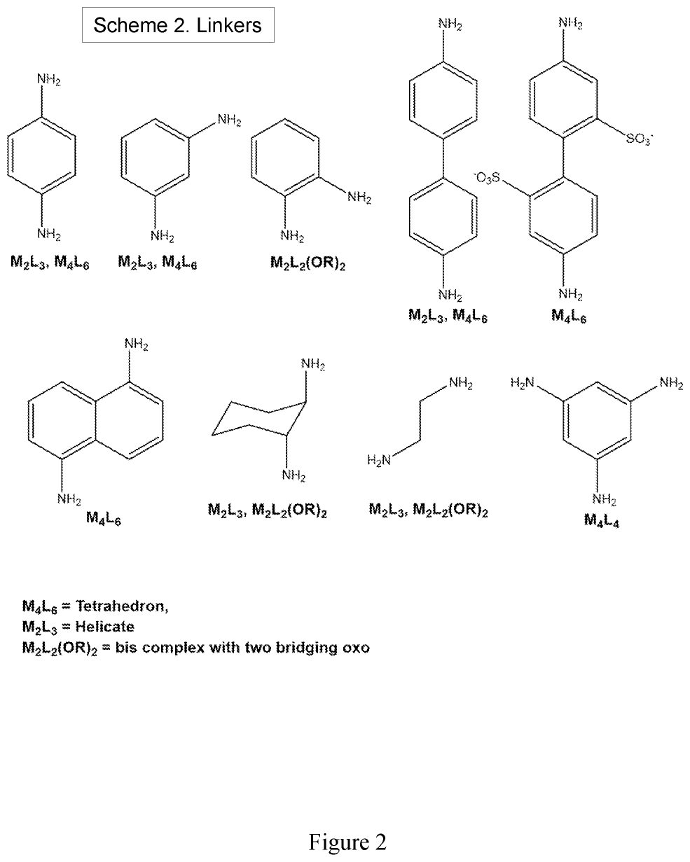

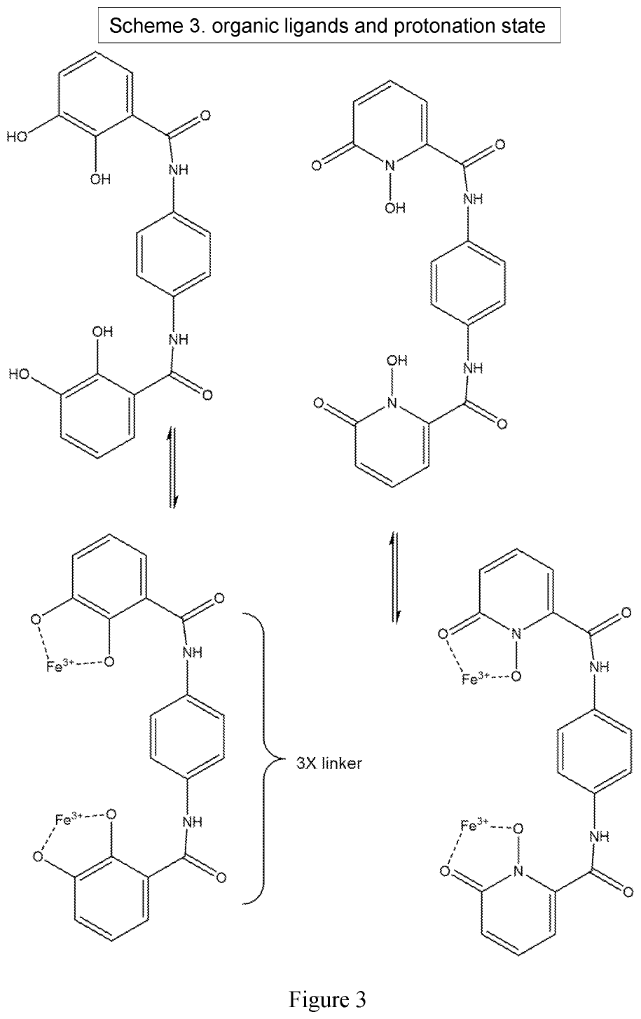

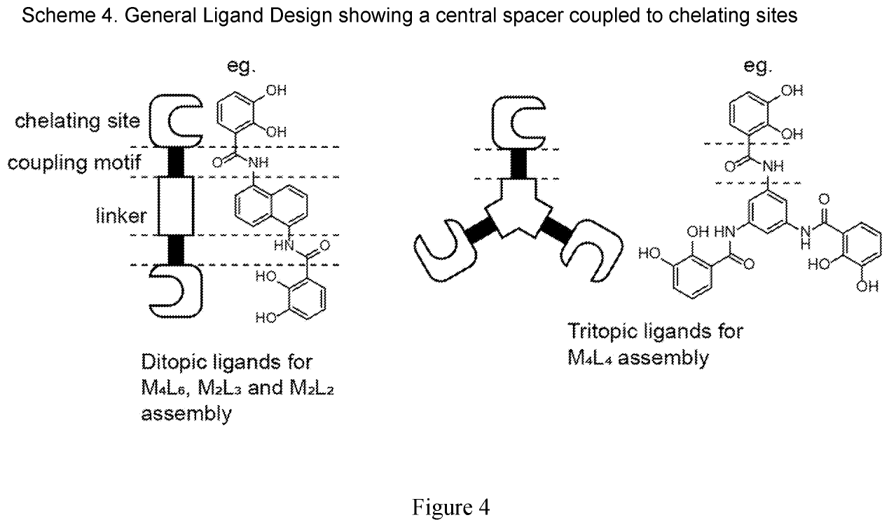

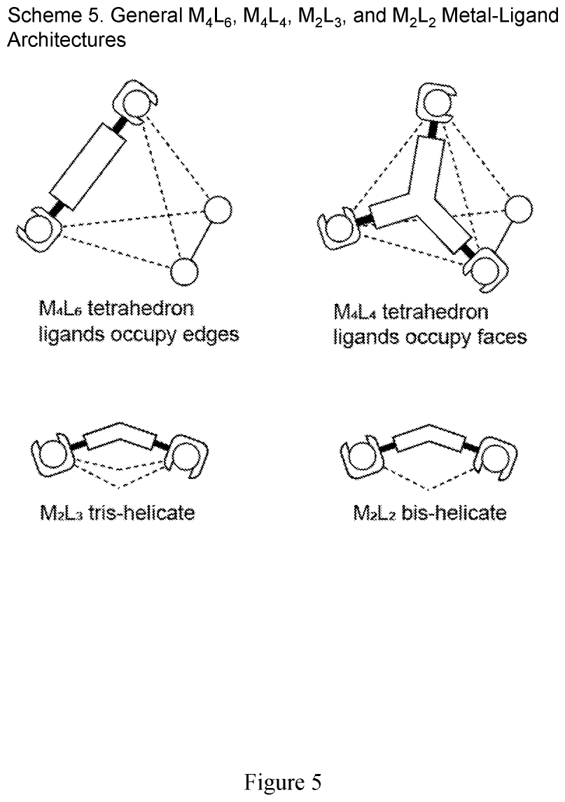

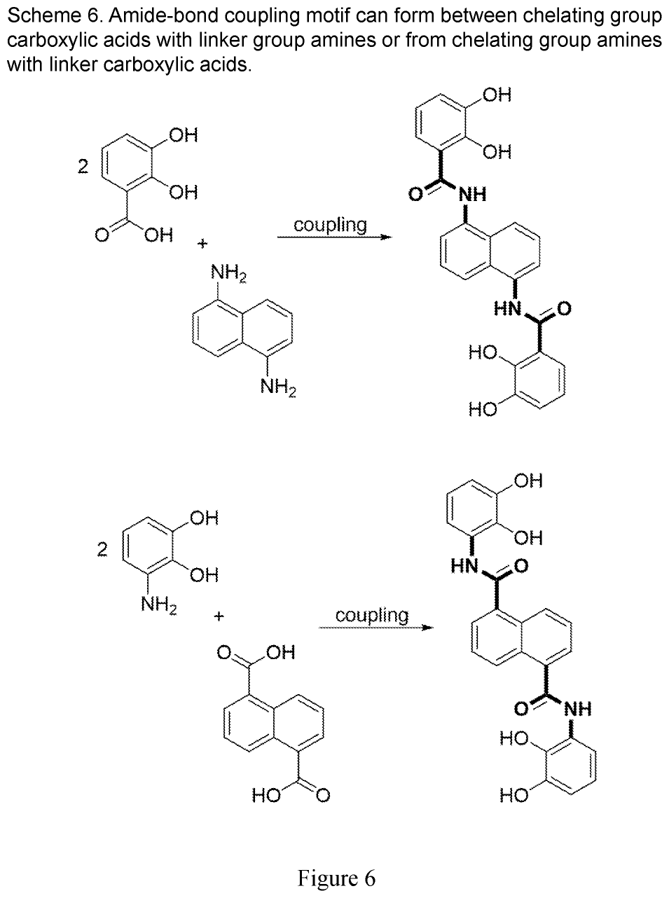

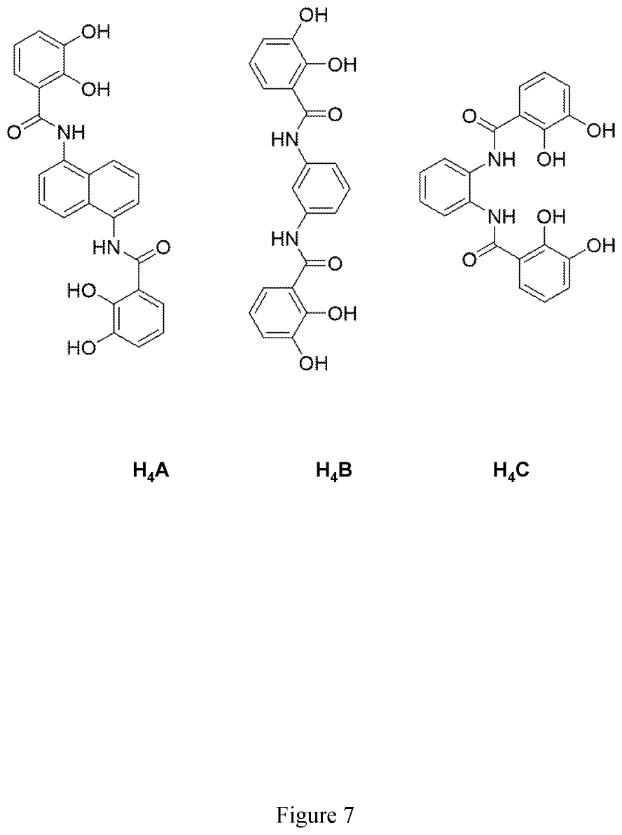

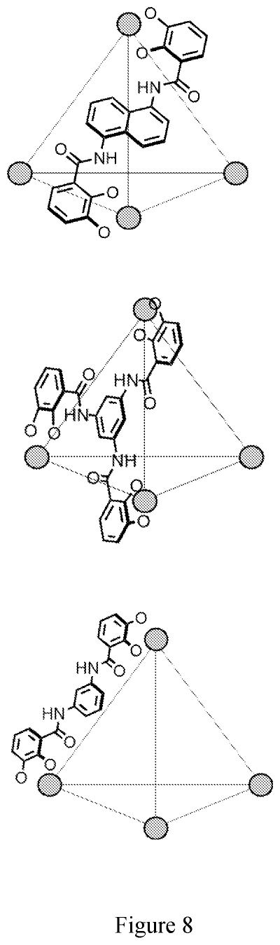

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range. As an illustrative example, any range provided herein includes all values that fall within the ranges to the tenth decimal place, unless indicated otherwise.

As used herein, unless otherwise stated, the term “group” refers to a chemical entity that has one terminus or two or more termini that are covalently bonded to one or more other chemical spec(ies). The term “group” includes radicals (e.g., monovalent and multivalent, such as, for example, divalent, trivalent, and the like, radicals). Examples of groups include, but are not limited to:

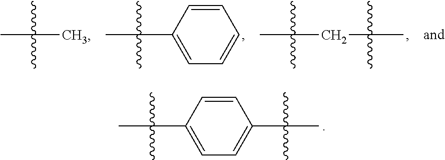

As used herein, unless otherwise indicated, the term “alkyl” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, an alkyl group is a C₁ to C₁₂ alkyl group, including all integer numbers of carbons and ranges of numbers of carbons therebetween (e.g., C₁, C₂, C₃, C₄, C₅, C₆, C₇, C₈, C₉, C₁₀, C₁₁, or C₁₂). The alkyl group may be unsubstituted or substituted with one or more substituent(s). Examples of substituents include, but are not limited to, substituents such as, for example, halogens (—F, —Cl, —Br, and —I), aliphatic groups (e.g., alkyl groups, alkenyl groups, alkynyl groups), aryl groups, alkoxide groups, amine groups, carboxylate groups, carboxylic acids, ether groups, alcohol groups, alkyne groups (e.g., acetylenyl groups), and the like, and combinations thereof.

As used herein, unless otherwise indicated, the term “aryl” refers to C₅ to C₁₄ (e.g., C₅, C₆, C₇, C₈, C₉, C₁₀, C₁₁, C₁₂, C₁₃, or C₁₄), including all integer numbers of carbons and ranges of numbers of carbons therebetween, aromatic or partially aromatic carbocyclic groups. The aryl groups may comprise (or be) polyaryl groups such as, for example, fused ring or biaryl groups. The aryl group may be unsubstituted or substituted with one or more substituent(s). Examples of substituents include, but are not limited to, substituents such as, for example, halogens (—F, —Cl, —Br, and —I), aliphatic groups (e.g., alkenes, alkynes), aryl groups, alkoxides, carboxylates, carboxylic acids, ether groups, sulfonic acids/sulfonates (which may be present as a salt such as, for example, a Group I cation, Group II cation, ammonium salt, or the like, or a combination thereof) groups, and the like, and combinations thereof. Examples of aryl groups include, but are not limited to, phenyl groups, biaryl groups (e.g., biphenyl groups), and fused ring groups (e.g., naphthyl groups).

As used herein, unless otherwise indicated, the term “heteroaryl” refers to a C₅ to C₁₄ (e.g., C₅, C₆, C₇, C₈, C₉, C₁₀, C₁₁, C₁₂, C₁₃, or C₁₄), including all integer numbers of carbons and ranges of numbers of carbons therebetween, monocyclic, polycyclic, or bicyclic ring groups (e.g., aryl groups) comprising one or two aromatic rings containing at least one heteroatom (e.g., nitrogen, oxygen, and sulfur atom) in the aromatic ring(s). The heteroaryl groups may be substituted or unsubstituted. Examples of substituents include, but are not limited to, substituents such as, for example, halogens (—F, —Cl, —Br, and —I), aliphatic groups (e.g., alkenes, alkynes), aryl groups, alkoxides, carboxylates, carboxylic acids, ether groups, sulfonic acid/sulfonate groups, and the like, and combinations thereof. Examples of aryl groups include, but are not limited to, phenyl groups, biaryl groups (e.g., biphenyl groups), and fused ring groups (e.g., naphthyl groups). Examples of heteroaromatic groups include, but are not limited to, benzofuranyl groups, thienyl groups, furyl groups, pyridyl groups, pyrimidyl groups, oxazolyl groups, quinolyl groups, thiophenyl groups, isoquinolyl groups, indolyl groups, triazinyl groups, triazolyl groups, isothiazolyl groups, isoxazolyl groups, imidazolyl groups, benzothiazolyl groups, pyrazinyl groups, pyrimidinyl groups, thiazolyl groups, thiadiazolyl groups, and the like, and combinations thereof.

The present disclosure provides compounds and ligands. The present disclosure also provides methods of making the compounds and ligands and uses of the compounds.

Compounds of the present disclosure provide an alternative to Gd(III) contrast agents. Without intending to be bound by any particular theory, it is considered that the compounds, which may be iron-based MRI contrast agents, produce contrast by the same paramagnetic mechanism as Gd(III) agents and are in small molecule form as coordination complexes, i.e., they are not nanoparticles. Potential advantages of using Fe(III) may include the extensive mechanisms in the human body for recycling and storage of iron, as the most abundant of the transition metal ions. Also, the redox potential of the Fe(III) complexes can be tuned to prevent reactive oxygen species (ROS) production. For example, certain ligands form redox-inactive Fe(III) complexes that do not produce hydroxyl radicals even under harsh conditions. Additionally the Fe(III) compounds may provide increased T₁ relaxivity (r₁ in mM⁻¹s⁻¹) and/or T₂ relaxivity (r₂ in mM⁻¹s⁻¹) values in blood serum and in vivo studies. It is desirable to obtain r₁ relaxivity values that are greater than clinically relevant Gd(III) contrast agents, such as, for example, Magnevist, of 3 mM⁻¹s⁻¹ at 37° C. at 4.7 Tesla and neutral pH. It is desirable to obtain r₁ relaxivities that are greater than those of Magnevist at field strengths of 0.5 to 4.7 Tesla (e.g., at an intermediate field strength of 3 to 4.7 Tesla). Also, in various examples, the compounds are desirable relaxivity agents that shorten the T₁ times of protons in water.

Compounds of the present disclosure can provide desirable control over spin and/or oxidation state of the Fe(III) cations in the molecules and/or desirable interactions of the molecules with inner-sphere and outer-sphere water. The compounds may stabilize Fe(III) in high-spin form. Also, the compounds may exhibit desirable aqueous solution chemistry.

In an aspect, the present disclosure provides compounds and ligands. The compounds may be referred to as metal organic polyhedra, supramolecular clusters, complexes, or self-assembled cages. The compounds may be self-assembled iron(III) or gallium(IIII) molecules. A compound, which may be a self-assembled iron(III) or gallium(III) molecule, may be made by a method of the present disclosure. Non-limiting examples of compounds and methods of making the compounds and uses of the compounds are provided herein.

Iron is present in the compounds in its trivalent form (Fe(III)). The Fe(III) may be high-spin Fe(III). The presence of high-spin Fe(III) may be determined by methods known in the art. For example, the presence of high-spin Fe(III) is determined using the Evans method (which may be carried out in solution). Gallium is present in the compounds in its trivalent form (Ga(III)). Ga(III) shows similar coordination chemistry to Fe(III) and the two ions may be used interchangeably if the trivalent state is maintained.

Compounds of the present disclosure may comprise one or more coordinatively saturated or coordinatively unsaturated Fe(III) cation(s), or a combination of coordinatively saturated or coordinatively unsaturated Fe(III) cations, which individually may be coordinatively saturated or coordinatively unsaturated high-spin Fe (III) cations. In certain cases, an individual coordinatively unsaturated high-spin Fe (III) cation has a coordination site to which a water molecule or a hydroxide ligand, or an alkoxide ligand (e.g., C₁ to C₁₂ alkoxide ligand) is coordinated that enhances their efficacy as T₁ MRI contrast agents.

As a non-limiting illustrative example, a compound has four iron(III) cations, which may be high-spin iron(III) cations, and/or has a tetrahedral structure and the compound is coordinatively saturated and has no coordinated (inner-sphere) water molecule(s) and/or hydroxyl ligand(s).

A compound comprises iron(III) cations, one or more or all of which may be high-spin iron(III) cation(s), gallium(III) cations, which may independently be a naturally-occurring stable gallium isotope or a radioactive gallium isotope (e.g., ⁶⁸Ga, which is a positron emitter)), or a combination of one or more such iron(III) cation(s) and one or more such gallium(III) cation(s). In various examples, the compounds are paramagnetic with two or more high-spin Fe(III) cations or diamagnetic Ga(III) compounds. The Ga(III) compounds may be studied by ¹H NMR spectroscopy.

A compound may comprise at least two structural units (which may be referred to as ligands, L, or A (e.g., H₄A), B (e.g., H₄B), C (e.g., H₄C), D (e.g., H₄D), E (e.g., H₄E), F (e.g., H₆F), G (e.g., H₆G), J (e.g., H₄J), L₁ (e.g., p-L₁H or b-L₁H or o-L₁H), L₂ (e.g., L₂H), or L₃ (e.g., m-L₃)). Each structural unit may have the same structure. One or more of the structural unit(s) may have a different structure relative to one or more or all of the other structural unit(s). A compound may comprise two or more iron(III) cations, two or more gallium(III) cations, or a mixture of one or more iron(III) cation(s) and one or more gallium(III) cation(s), where one or more or all of the iron(III) cation(s) are high-spin iron(III) cation(s). In certain examples, the ligands occupy one or more edge(s) of a polyhedron and the iron(III) cation(s), one or more of which may be high-spin iron(III) cation(s), gallium(III) cation(s), or a combination thereof each occupy one or more vertice(s) of the polyhedron. In certain examples, the ligands occupy one or more face(s) of a polyhedron and the iron(III) cation(s), one or more of which may be high-spin iron(III) cation(s), gallium(III) cation(s), or a combination thereof each occupy one or more vertice(s) of the polyhedron.

Various structural units can be used. Each structural unit may comprise at least one spacer group (which may be referred to a linker group) and two or more donor groups (which may be referred to as chelating groups and may have one or two groups that coordinate to an iron(III) cation), and at least two iron(III) cations (which may be high-spin iron(III) cations).

Ligands may have predefined geometries that can impart specific constraints to the metal complex coordination geometry while possessing rigid or restrained conformations to form self-assembled clusters of desired MOPs. For example, tris and bis catechol complexes of Fe(III) and Ga(III) are known to form complexes possessing D₃ symmetry with a C₃ axis of rotation through the metal center. These metal clusters form the vertices of the tetrahedrons as the geometric shape has the same axis of rotation.

The geometry of the self-assembled cluster may also be influenced by the spacer used to bridge donor groups (e.g., HOPO donors, catecholate donors, and the like). Planar species, which have a C₂ axis of rotation, with two amide linkages will form clusters with ratios of metal (M) to ligand(L) of M₂L₃ and M₄L₆ while a C₃ axis of rotation will form clusters of M₄L₄. When using spacers with a C₂ axis of symmetry it is possible to obtain mixtures of both M₂L₃ and M₄L₆ with an entropically favored M₂L₃ cluster. In order to prevent the formation of M₂L₃ clusters, the spacer amides should have an offset from the central axis introducing a steric hindrance to the formation of M₂L₃ clusters resulting in the selective formation of M₄L₆ clusters. M₂L₂ double helicate clusters can be formed by either stoichiometric control of the metal to ligand ratio or through specific selection of spacers preventing a triple helicate structures.

A compound (or ligand) may comprise various donor groups. A donor group has one or more functional group(s) that can coordinate to an iron(III) cation or a gallium(III) cation.

It is desirable that the ligand donors stabilize the iron and/or gallium cation(s) spin state and/or oxidation state. Without intending to be bound by any particular theory it is considered that ligand donors that are polydentate ligands, which may form, for example, bis and tris metal complexes while stabilizing the desired metal spin state and/or oxidation state, are desirable.

A compound (or ligand) may comprise various spacer groups. A spacer group has at least two covalent bonds, where each covalent bond is to a donor group.

Donor groups and spacer groups may be covalently bound together by various groups. These groups may be referred to as linking groups. If a structural unit comprises aromatic groups, desirable linking groups preserve the planar character of the aromatic system of the structural unit. As an illustrative example, amide coupling is desirable for bridging the aromatic donors and spacers as it preserves the planar character of the aromatic system while hydrogen bonding through the hydroxyl catechol donor with the amide linkage pre-organizes the ligand for self-assembly. Additionally, amide bonds are robust to hydrolysis under physiological pH and are thus suitable for ligands designed for in vivo use.

The spacer group(s) and/or donor group(s) may have one or more substituent(s) (e.g., R₁ group). The spacer group(s) and/or donor group(s) may comprise (or be) an aryl group/groups and/or a heteroaryl group/groups and the substituent(s) may be on an individual aryl group and/or individual heteroaryl group (e.g., the aryl ring of the aryl group and/or the nitrogen atom of a heteroaryl group). The substituent(s) may be chosen from alkyl groups, aryl groups, heteroaryl groups, polyether (PEG) groups, sulfonic acid/sulfonate, targeting groups, or the like, or a combination thereof.

Polyethylene group (PEG) groups may be attached to a spacer group or a donor group through terminal carboxylic acid, thiol, azide, amine, or alcohol functional groups. The PEG groups may contain 1 to 1000 (e.g., 1 to 100) ethylene glycol repeat units (e.g., one, two, four, five, six, twelve, eighteen, five-hundred, six-hundred, seven-hundred-fifty or one-thousand repeat ethylene glycol units), including all integer number of repeat units and ranges therebetween.

Various ligands, spacer groups, and donor groups are described herein. A ligand or donor group may be depicted in a protonated (e.g., fully or partially protonated form). The depiction of any such ligand or donor group herein includes the fully or partially protonated form, which may be the form of the ligand or donor group that is coordinated to an iron(III) cation and/or gallium(III) cation. A spacer group may be depicted in a form that is not covalently bound to one or more donor groups (s) or a donor group may be depicted in a form that is not covalently bound to one or more spacer(s). The depiction of any such spacer group or donor group herein includes a covalently bound form of the spacer group or donor group.

A compound may comprise various targeting groups. Non-limiting examples of targeting groups include proteins, peptides, antibodies, aptamers, small molecules that bind to cell receptors such as, for example, folate, or small molecule antagonists, and the like, and combinations thereof.

The spacer group may comprise (or be) an aryl group and/or a heteroaryl group. One or more of the aryl group(s) of an aryl spacer group and/or heteroaryl group of the heteroaryl group may be substituted with one or more sulfonic acid and/or sulfonate group(s).

In various examples, the following spacer provides Fe(III) compounds that are coordinatively unsaturated:

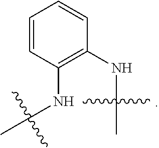 [SMILES: *Nc1ccccc1N*]

The coordinatively unsaturated Fe(III) compounds may have one or more other monodentate ligand(s) and/or one or more other ligand(s) bridging two Fe(III) centers

For 1,2-HOPO donors, it may be desirable for one or more spacer group(s) to have an anionic substituent, such as, for example, a sulfonyl group (RSO₃⁻, where R is an alkyl group (e.g., a C₁ to C₁₂ alkyl group (e.g., C₁, C₂, C₃, C₄, C₅, C₆, C₇, C₈, C₉, C₁₀, C₁₁, or C₁₂)), or the like. Without intending to be bound by any particular theory, it is considered that the anionic substituent(s) increase solubility, for example, as shown in pS-L3 or b-L1H. The HOPO type ligands loose just one proton to give monoanionic donors bound to M, where the catechol groups lose two protons to give dianionic groups for Fe(III) or Ga(III). Thus, the HOPO derivatives may benefit from extra anionic charge the linker so that the overall charge on the MOP is not neutral.

In various examples, the compounds (or ligands) of the disclosure are a salt, a partial salt, a hydrate, a polymorph, or a stereoisomer, or a mixture thereof. For example, a compound is a racemic mixture, a single enantiomer, a single diastereomer, or mixture of diastereomers. In certain examples, the compounds mixtures of diastereomers and/or conformers which can be determined by, for example, NMR, crystal structure analysis, and the like. The diastereomers may arise from the conformation of the compound and/or the directionality of one or more of the substituent(s) on a structural unit or units of the compound.

A compound (or a ligand) may be a salt or partial salt. The compound may comprise one or more cation(s) (e.g., be isolated as a salt or partial salt). In the case of compounds with more than one cation, the cations may be the same or one or more of the cations may be different than one or more or all of the other cations. Non-limiting examples of cations include Na⁺, K⁺, NH₄⁺, NR₄⁺ (where R is an alkyl group (e.g., C₁ to C₁₂ alkyl group (e.g., C₁, C₂, C₃, C₄, C₅, C₆, C₇, C₈, C₉, C₁₀, C₁₁, or C₁₂)) or H group), PR₄⁺ (where R is an alkyl group (e.g., C₁ to C₁₂ alkyl group (e.g., C₁, C₂, C₃, C₄, C₅, C₆, C₇, C₈, C₉, C₁₀, C₁₁, or C₁₂)) or H group), Mg²⁺, Ca²⁺, and the like, and combinations thereof. As an illustrative example, the compound below may be isolated as the sodium salt (e.g., Na₄[Fe₂C₂(OMe)₂]). In other words, the compound has a minus four charge and requires four monovalent cations to be electrically neutral.

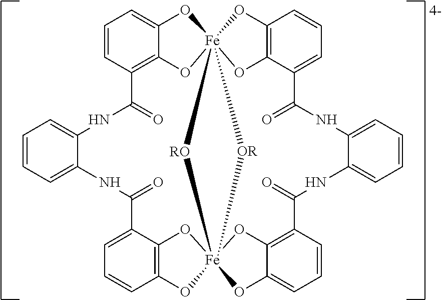

As an illustrative example, the compound below may be isolated as the ammonium salt (e.g., (NH₄)₁₂[Fe₄A₆]). In other words, the compound has a minus twelve charge and requires twelve monovalent cations to be electrically neutral.

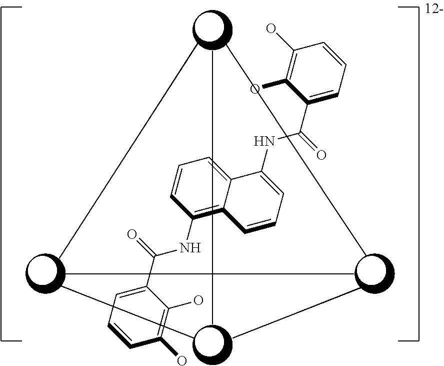

The compounds of the present disclosure may be thermodynamically stable and/or kinetically inert towards dissociation. In an example, the kinetic inertness of the compounds of the present disclosure can be described using a rate constant for dissociation. In an example, the structural units do not dissociate appreciably from the metal center(s) (e.g., 1% or less, 0.1% or less, or 0.01% or less dissociation is observed) for 2 hours or more, 6 hours or more, 12 hours or more, 24 hours or more, or 48 hours or more at a neutral pH (e.g., a physiological pH, for example, about pH 7 (e.g., pH 7.2)), in the presence of 1) 25 mM carbonate, 0.40 mM phosphate, 100 mM NaCl; or 2) 100 mM NaCl.

In an example, one or more or all of the Fe(III) cation(s) are high-spin S=5/2. A paramagnetic spin state may provide desirable T₁ (longitudinal) relaxation of water protons. In order to keep Fe(III) in the high-spin state, the ligand (or crystal) field splitting must not be too large. If the crystal field splitting is larger than the pairing energy, a low spin (S=½) state will result. Fe(III) is maintained in a high-spin paramagnetic state with a range of structural units.

In various examples, the compound (e.g., a compound having an M₄L₆ (e.g., with first two spacer groups below), M₂L₂ (e.g., with the fourth spacer group below), M₂L₃ (e.g, with the third spacer group below), or M₄L₄ (e.g., with the fifth/last spacer group below) structure) does or does not comprise only gallium(III) cations and only one of the following spacer groups and two or more (e.g., two or three) catechol groups:

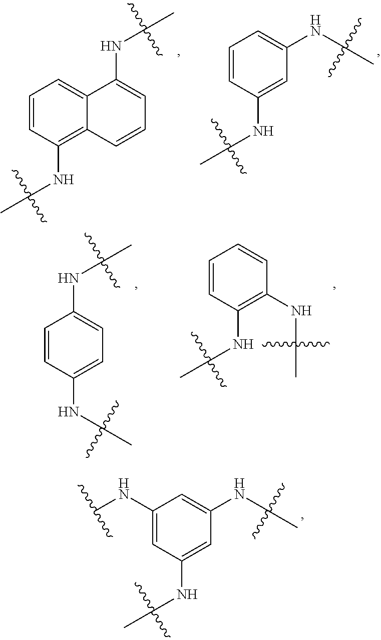 [SMILES: *Nc1cccc2c(N*)cccc12 ; *Nc1cccc(N*)c1 ; *Nc1ccc(N*)cc1 ; *Nc1ccccc1N* ; *Nc1cc(N*)cc(N*)c1]

where each individual spacer group is covalently bound to at least one catechol group via an amino group of the donor group.

In various examples, the compound (e.g., a compound having an M₄L₆ (e.g., with the first spacer group below) or M₂L₃ (e.g., with the second spacer group below) structure) does or does not comprise only iron (III) cations, which may be high-spin iron(III) cations, and only one of the following spacer groups and one or more catechol group(s):

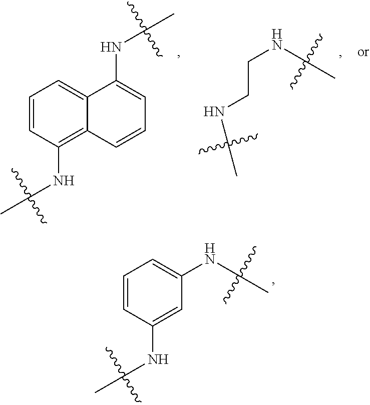 [SMILES: *Nc1cccc2c(N*)cccc12 ; *NCCN* ; *Nc1cccc(N*)c1]

where an individual spacer group is covalently bound to at least one catechol group via an amino group of the donor group.

A compound may have an MₓL_{y} formula, where each M is Fe(III) or Ga(III) as described herein and L is a ligand as described herein. In various examples, the compound is chosen from:

M₂L₂ (e.g., where L is C, E, J, or o-L₁ and M is independently chosen from Fe(III) and Ga(III));
M₂L₃ (e.g., where L is b-L₁, p-L₁, p-L₂ or D and M is independently chosen from Fe(III) and Ga(III));
M₄L₄ (where L is G or F and M is independently chosen from Fe(III) and Ga(III)); and
M₄L₆ (e.g., L is A or B or n-L₁ and M is independently chosen from Fe(III) and Ga(III)), where the specific ligands (L) are

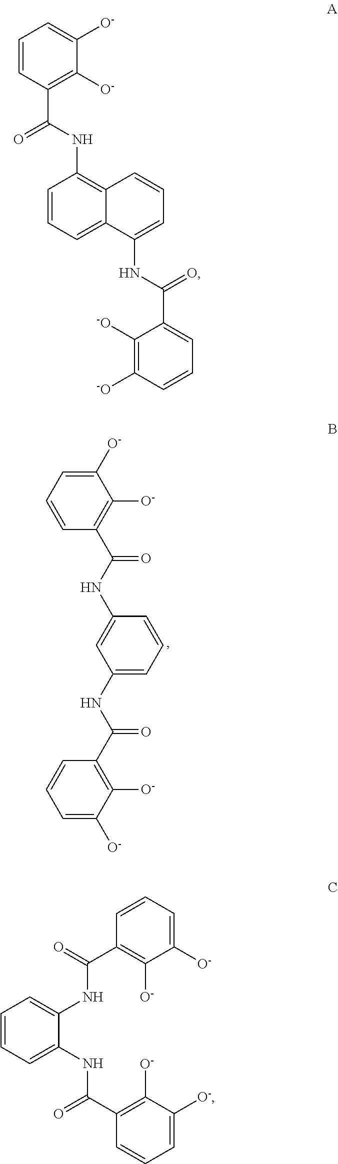

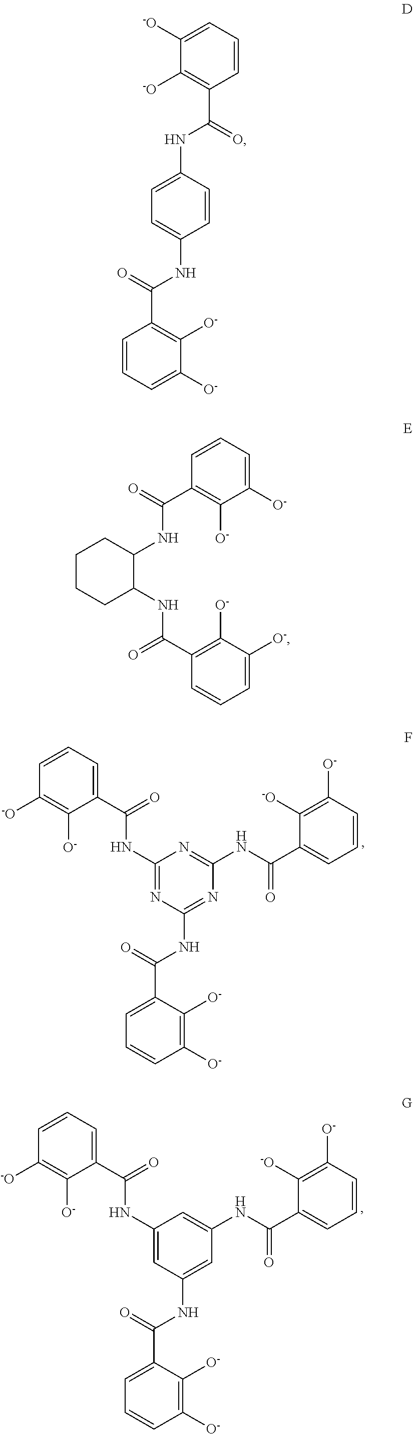

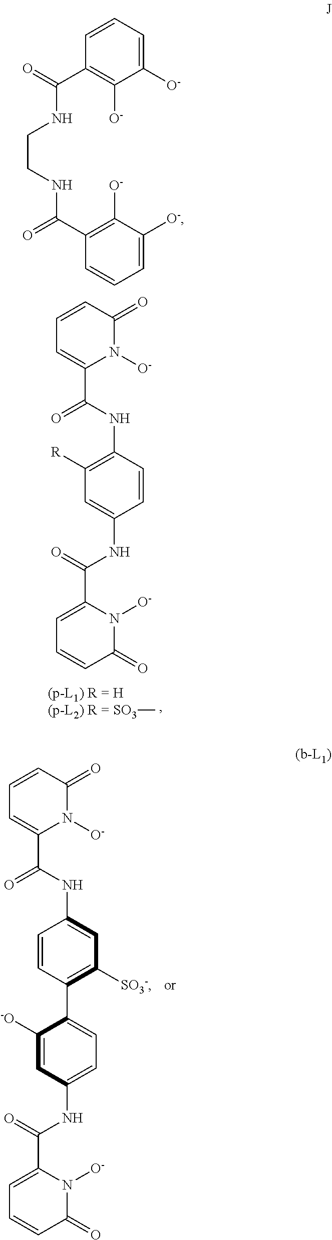

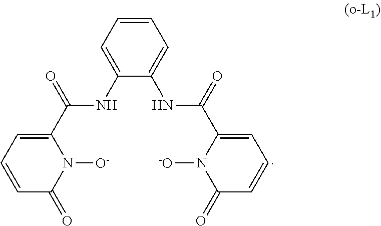

In various examples, the compound is not Fe₄A₆, Ga₄A₆, Ga₄B₆, Ga₂D₃, or Fe₄(m-L₃)₆ formula, where the ligands (e.g., Ls) are

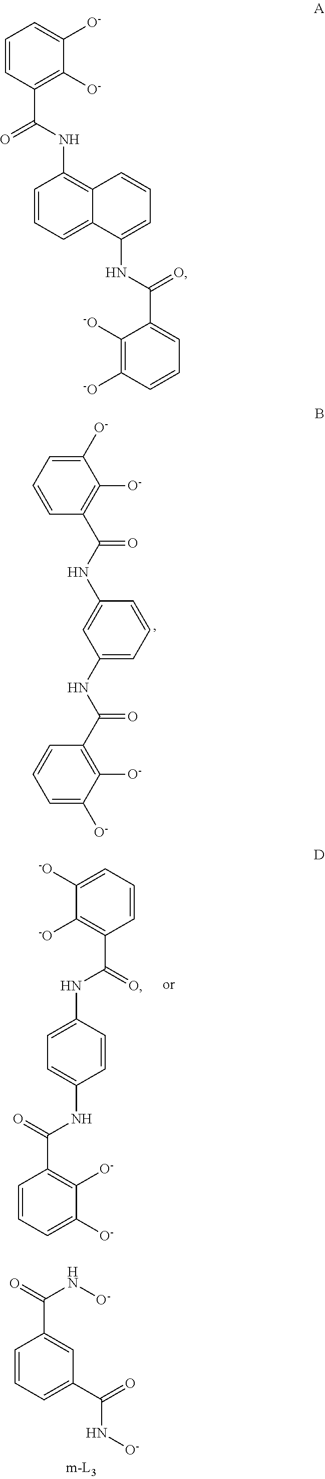

In various examples, a ligand is or a compound comprises one or more ligand(s) chosen from the following:

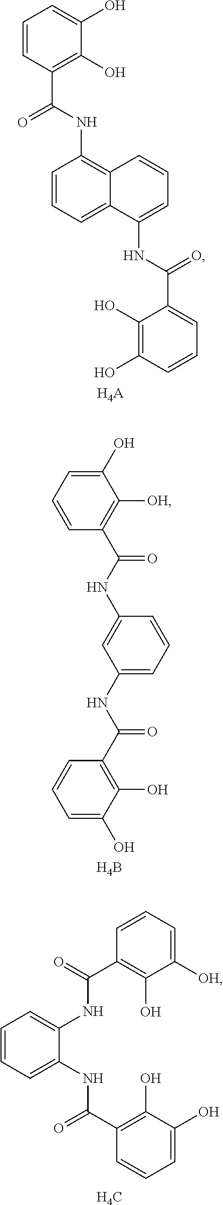

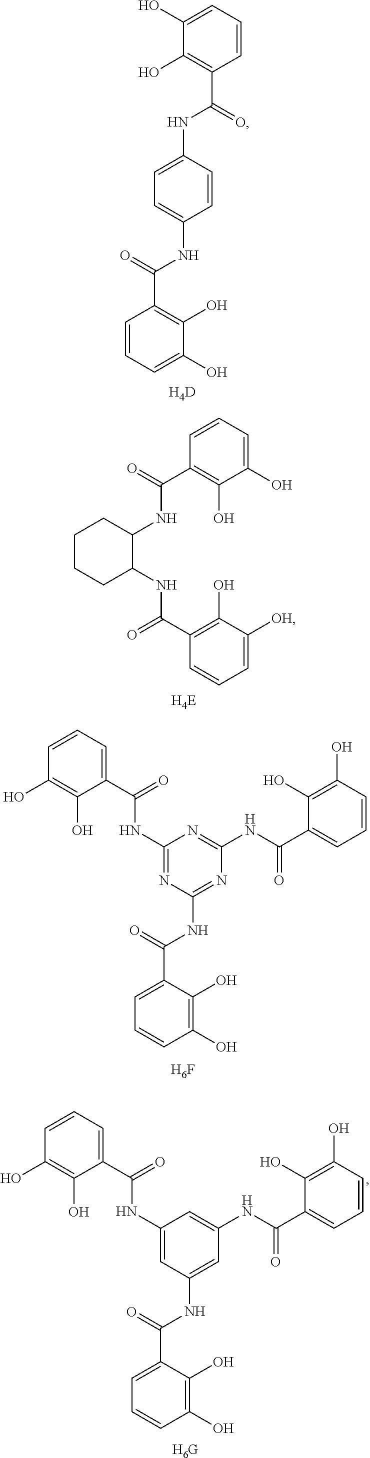

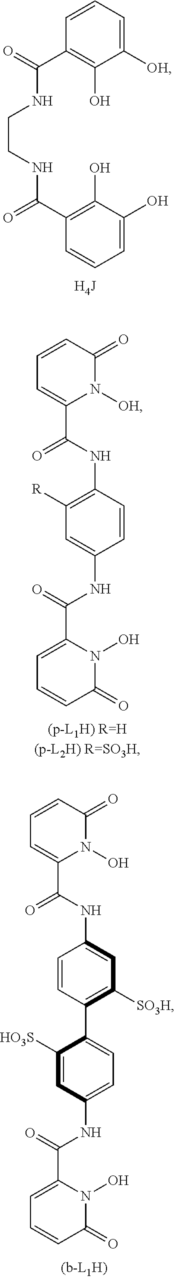

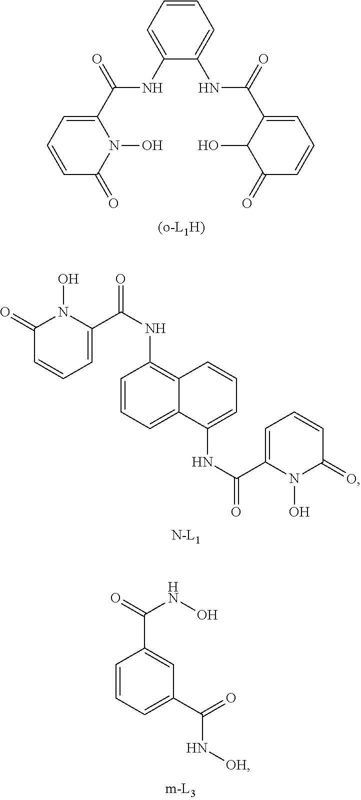 [SMILES: O=C(Nc1ccccc1NC(=O)c1cccc(=O)n1O)C1=CC=CC(=O)C1O ; O=C(Nc1cccc2c(NC(=O)c3cccc(=O)n3O)cccc12)c1cccc(=O)n1O ; O=C(NO)c1cccc(C(=O)NO)c1]

or a partially or completely deprotonated analog (e.g., to form one or more—O⁻ group(s)) thereof. In various examples, the compound comprises or does not comprise only one of these ligands.

It is desirable that the ratio of the enhanced shortening of T₁ to T2 time constants also expressed as rate constants (R₁/R₂) of a compound of the present disclosure are close to one (unity). The R₂, or transverse relaxation rate constant given ins⁻¹, is by definition always larger than R₁, the longitudinal relaxation rate constant. In various examples, Fe(III) contrast agents of the present disclosure have desirably low R₂ to give R₁/R₂ ratios close to one. In various examples, a complex or compound of the present disclosure have R₁/R₂ ratios of 0.8 to 0.1, 0.5 to 0.1, 0.3 to 0.1, or 0.8 to 0.6.

Without being bound by any particular theory, it is considered that exchange of inner-sphere water with bulk water is an important mechanism for proton relaxivity. A compound may have a structural configuration such that inner-sphere and/or outer-sphere water interactions give a decrease in the T₁ relaxation times of bulk water protons. A compound may be high-spin Fe(III) under biologically reducing conditions and have a structural configuration such that inner-sphere and/or outer-sphere water interactions give a decrease in the T₁ relaxation times of bulk water protons.

There are several structural properties to improve relaxivity including consideration of inner-sphere waters, second-sphere waters, outer-sphere waters, and their exchange rates; correlation times for rotation of the entire molecule; and the electronic relaxation times. A compound may exhibit or have, as appropriate, one or more or all of these properties and/or structural features.

Each of these contributions may be varied and their importance depends on the magnetic field strength of the scan. Contributions to relaxivity are detailed below.

Inner-sphere ligands (q) contribute to relaxivity (r₁^{IS}) and second-sphere ligands also contribute (r₁^{SS}) as in Eq. 1 and Eq. 2.

$${r}_{1} = {r}_{1}^{S S} + {r}_{1}^{I S} \qquad Equation 1$$

$${r}_{1}^{IS} = \frac{q / [ {H}_{2} O ]}{{T}_{1 m} + {τ}_{m}} \qquad Equation 2$$

Eq. 1 shows that relaxivity has contributions from bound water (inner-sphere, IS) and second-sphere (SS) (outer-sphere) water. Eq. 2 predicts that greater numbers of bound water molecules and rapid ligand exchange rate constants (short lifetimes for bound water (τₘ) are advantageous. Notably r₁, the parameter used to characterize relaxivity, has units of mM⁻¹s⁻¹, and is obtained from a plot of T_{lobs} (s⁻¹) versus contrast agent concentration. There is an analogous relationship for second-sphere waters although the number and residence time is not well defined. There are analogous equations that define r₂ relaxivity.

Fe(III) complexes with no inner-sphere water molecules can effectively enhance relaxation of the protons of the water. Without intending to be bound by any particular theory, it is considered that exchange of inner-sphere water with bulk water is an important mechanism for proton relaxivity. However, Fe(III) is a much smaller metal ion than Gd(III) (0.78 Å vs. 1.25 Å, respectively). The shorter M−H distance in bound water of Fe(III) compared to Gd(III) suggests that the relative efficiency of the outer-sphere versus inner-sphere contributions may differ for the two metal ion complexes.

There are three mechanisms that contribute to paramagnetic relaxation of associated water (1/T₁ₘ): the scalar (contact) contributions, dipole-dipole contributions and Curie spin relaxation. The most important of these for the longitudinal relaxation considered here is the dipole-dipole contribution (1/T₁DD). At field strengths of 1.5 T or greater, 1/T₁DD is defined as shown in Eq. 3 where S is the spin quantum number, ω_{H} is the Larmor frequency of the proton, r_{MH} is the metal ion-proton distance and γ_{H} is the proton gyromagnetic ratio, gₑ is the electronic g factor, μ_{B} is the Bohr magneton, and μₒ is the permittivity of a vacuum. Notably, the 1/T₁DD term increases (higher relaxivity) with larger total spin (S) which favors Gd(III) over Fe(III). However, the shorter distance of the paramagnetic Fe(III) center to water protons (r_{MH}) favors Fe(III) proton relaxation, especially given the 1/r⁶ dependence.

$$\frac{1}{{T}_{1}^{D D}} = \frac{2}{15} ( \frac{{μ}_{0}}{4 π} ) \frac{{γ}_{H}^{2} {g}_{e}^{2} {μ}_{B}^{2} S ( S + 1 )}{{Γ}_{M H}^{6}} [ \frac{3 {τ}_{C}}{1 + {ω}_{H}^{2} {τ}_{C}^{2}} ] \qquad Equation 3$$

$$\frac{1}{{τ}_{C}} = \frac{1}{{τ}_{R}} + \frac{1}{{T}_{1 e}} + \frac{1}{{τ}_{m}} \qquad Equation 4$$

The correlation time (τc) for the dipolar relaxation mechanism is influenced by different processes including the lifetime of the bound water (1/τₘ), the rotational motion of the contrast agent (1/τ_{R}) and the longitudinal relaxation of the unpaired electrons (1/T₁ₑ). Although any of these three processes can contribute, their importance depends on the strength of the magnetic field. Much of the literature is focused on the importance of these processes at low field strength (<1 T). Under these conditions, the rotational processes or electronic relaxation times may be limiting, and τₘ should be in a narrow range close to 10 ns (kₑₓ=10⁸s⁻¹). However, at higher field strengths (≥1.5 T), simulations show that the optimal τₘ has a larger range (1-100 ns) and rotational motions should have values intermediate between small molecules and proteins. An important parameter is T₁ₑ, the electronic relaxation time. A long T₁ₑ for Fe(III) may result from complexes that have a high degree of symmetry, leading to little zero field splitting and slow relaxation of the electronic state.

Also, the shape, size, and rigidity of the molecule affect the rotational correlation time. In general, rigid molecules that do not have local rotational motion produce longer correlation times and higher relaxivity. Normally, the correlation time τ_{c} should match the Lamor frequency of the experiment. Thus, an approach to obtain higher relaxivity is to connect multiple paramagnetic centers together. This may be done with rigid linkers that bridge the metal ion centers with a high degree of constraint. It is also desirable that the linkers have the ability to undergo limited or no rotational motion.

In an aspect, the present disclosure provides compositions. A composition may comprise one or more compound(s) of the present disclosure. A composition may comprise one or more compound(s) of the present disclosure and a pharmaceutically acceptable carrier.

A composition may also comprise one or more protein(s). The protein(s) may associate with the compound. The protein(s) may be protein(s) that are found (e.g., are predominant) in the blood of an individual. Non-limiting examples of proteins include, human serum albumin, and the like, and combinations thereof.

For use in methods of the disclosure, the compounds described herein may be administered as pharmaceutical preparations. Accordingly, they may be provided in a variety of compositions, and may be combined with one or more pharmaceutically acceptable carrier(s). Non-limiting examples of pharmaceutically acceptable carriers can be found in REMINGTON'S PHARM. SCI., 15th Ed. (Mack Publ. Co., Easton (1975)), the disclosure of which with regard to pharmaceutically acceptable carriers is incorporated herein by reference. Non-limiting examples of materials which can be used as pharmaceutically-acceptable carriers include sugars, such as, for example, lactose, meglumine, glucose and sucrose; starches, such as, for example, corn starch and potato starch; cellulose, and its derivatives, such as, for example, sodium carboxymethyl cellulose, ethyl cellulose and cellulose acetate; powdered tragacanth; malt; gelatin; talc; excipients, such as, for example, cocoa butter and suppository waxes; oils, such as, for example, peanut oil, cottonseed oil, safflower oil, sesame oil, olive oil, corn oil and soybean oil; glycols, such as, for example, propylene glycol; polyols, such as, for example, glycerin, sorbitol, mannitol and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances typically employed in pharmaceutical formulations; and the like; and combinations thereof. The composition can be provided as a liquid, a solution, or a solid, and may be provided in combination with any suitable delivery form or vehicle, examples of which include, but are not limited to, caplets, capsules, tablets, an inhalant, an aerosol, etc.

In an aspect, the present disclosure provides methods of making self-assembled iron(III) or gallium(III) compounds. Examples of methods of making ligands and compounds of the present disclosure are provided herein.

A compound of the present disclosure may be prepared, for example, as described herein. The following examples are presented to illustrate the present disclosure. They are not intended to be limiting in any manner. Those skilled in the art will recognize that routine modifications to these examples can be made which are intended to be within the scope of the disclosure.

An illustrative, non-limiting example of method of making a structural unit/ligand of the present disclosure is described in Scheme 1.

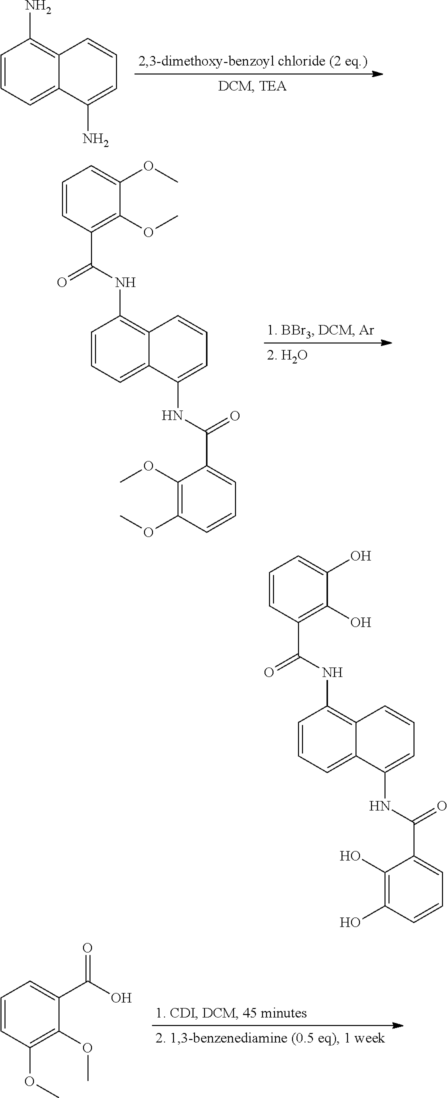

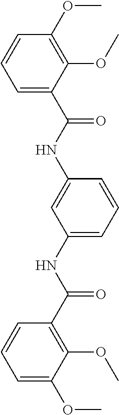

The methyl protecting groups on the catechol groups can be removed to provide a structural unit/ligand. This general scheme can be used to synthesize a variety of structural units/ligands of the present disclosure.

An illustrative, non-limiting example of method of making a structural unit/ligand and compound of the present disclosure is described in Scheme 2A and B.

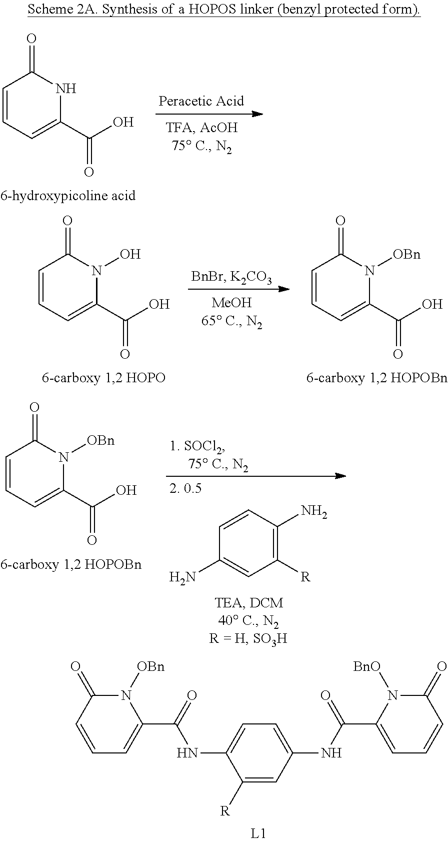

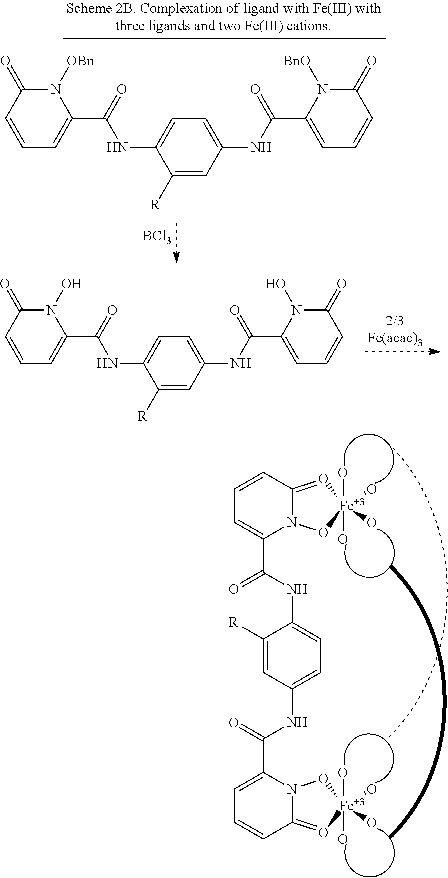

The solid and dashed lines represent additional complexed ligands.

General synthesis of an illustrative, non-limiting example of general synthesis that can be used to make a compound of the present disclosure is described in Scheme 2.

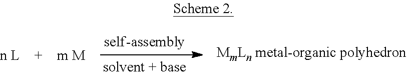

General synthetic method for the formation of Fe(III) and/or Ga(III) metal-organic polyhedrons: A ligand or mixture of ligands is dissolved in an appropriate organic solvent (e.g. water, methanol, ethanol). To this ligand solution is added a suitable base (e.g. KOH, NaOH, potassium carbonate). A source of trivalent metal ion or ions (e.g., Fe(III) cations and/or Ga(III) cations) is added either as a solid or dissolved in a separate solution. The stoichiometry of ligand (n) and metal (m) matches the ratio in the final metal-organic polyhedron. For example, an M₄L₆ tetrahedron is formed from six equivalents of ligand (n=6) and four equivalents of trivalent metal precursor (m=4). For the M₄L₄ face-directed species, n=4 and m=4. For the M₂L₃ triple helicates, n=3 and m=2. For metal-organic polyhedrons containing two or more types of ligands and/or both Ga(III) and Fe(III) ions, the total number of ligand and metal equivalents are represented by n and m, respectively. The self-assembly solution is stirred at room temperature, for example, for 1-24 hours, such as, for example, for 1-2 hours, 2-4 hours, 4-6 hours, 6-12 hours, or 12-24 hours). The metal-organic polyhedrons precipitate from solution and may be collected by filtration or centrifugation.

In an aspect, the present disclosure provides uses of compounds of the present disclosure. The compounds may be used in imaging methods.

The imaging methods of the present disclosure can be used to image a cell, tissue, organ, vasculature, or a part thereof. The cell, tissue, organ, vasculature may be a part of an individual. By “individual”, it is meant a human or non-human animal (e.g., cow, pig, mouse, rat, cat, dog, or other agricultural, pet, or service animal, and the like). In an example, the disclosure provides a method to obtain an image of at least a portion of a cell, tissue, organ, or vasculature comprising the steps of: contacting a cell, tissue, organ, or vasculature with one or more compound(s) of the present disclosure, and imaging at least a portion of the cell, tissue, organ, or vasculature to obtain an image of the portion of cell, tissue, organ, or vasculature. At least part of a cell, tissue, or organ may be alive or dead. Likewise, the individual can also be alive or deceased.

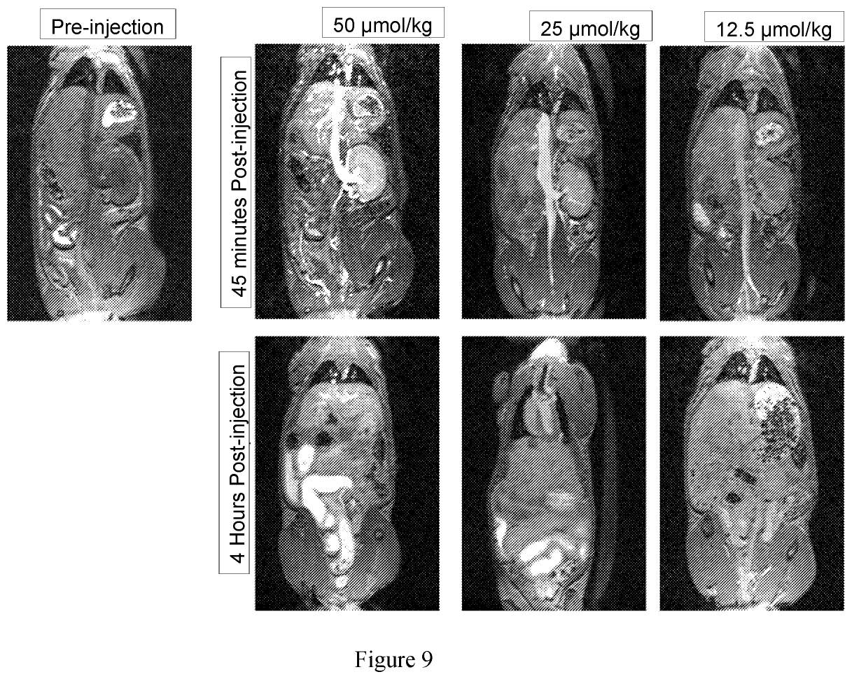

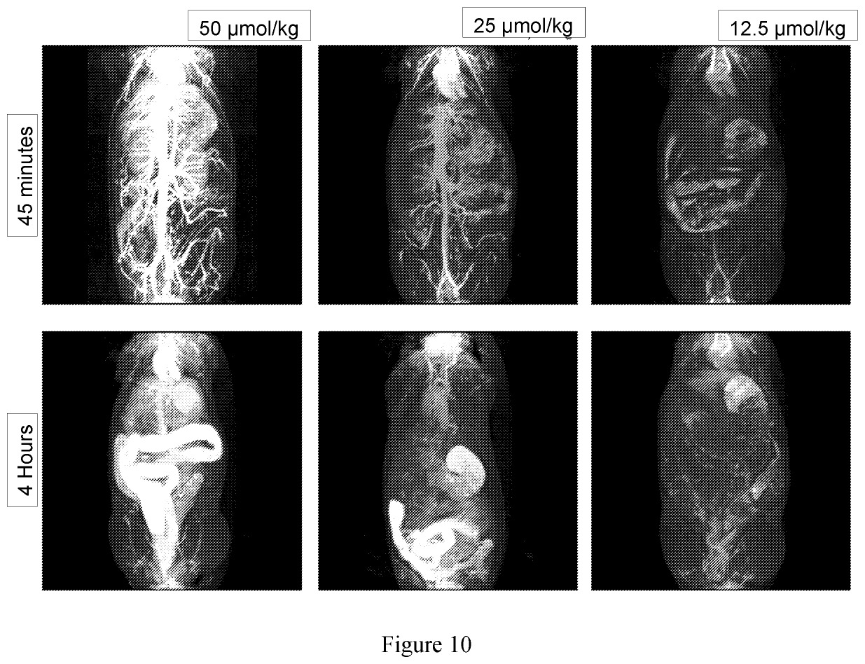

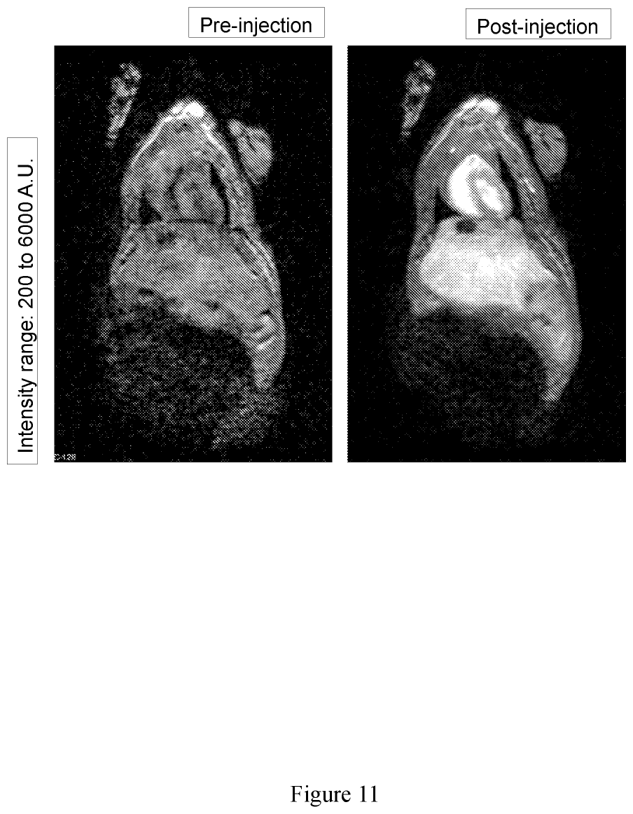

A compound or compounds comprising one or more Fe(III) cations may be used as an Fe(III) T₁ MRI contrast agent or agents. The contrast is produced by T₁ weighted imaging to give positive contrast in the region where the iron complexes accumulate. FIGS. 9-11 show data from in vivo MRI studies done in mice.

The compound(s) may be administered as pharmaceutical preparations. Accordingly, they can be provided in a variety of compositions, and may be combined with one or more pharmaceutically acceptable carrier(s) (e.g., as described herein).

Various methods known to those skilled in the art may be used to introduce the compositions of the disclosure to an individual. These methods include, but are not limited to, intravenous, intramuscular, intracranial, intrathecal, intradermal, subcutaneous, and oral routes. In an example, the composition is administered intravenously.

The necessary solubility of the complexes contributes to their effectiveness in producing contrast. For Fe(III) T₁ contrast agents that produce good water proton relaxivity, it is desirable that the complexes exhibit at least 5 mM solubility. However, other additives such as, for example, human serum albumin (HSA) or meglumine may be used to increase solubility and/or increase relaxivity. Addition of HSA (e.g., 35 mg/mL) to some of the Iron(III) complexes produces higher T₁ relaxivity as shown in Table 1 and 2). Solubility may be measured in aqueous solution at near neutral pH (e.g., 6.5 to 7.5, including all 0.1 pH values and ranges therebetween) in 100 mM NaCl with 25 mM carbonate and 0.4 mM phosphate.

The dose of the compound to be used will typically be dependent upon the needs of the individual to whom the compound of the disclosure is to be administered. These factors include, but are not necessarily limited to, the weight, age, sex, and medical history of the individual.

It is desirable that the Fe(III) cation(s) of a compound or the Ga(III) cation(s) of a compound remain in the trivalent oxidation state and not be reduced by, for example, peroxide, superoxide, ascorbate, or by glutathione at concentrations present in the extracellular medium of cells such as, for example, mammalian cells (e.g., human cells). Normally, a redox potential more negative than zero mV (<0 mV) versus NHE is sufficient. If the complex were to be reduced to Fe(II) and the Fe(II) complex and the complex has a positive redox potential versus NHE, reactive oxygen species may be produced.

In various examples, a compound or compound of the present disclosure exhibits a reduction potential (Eₒ) of less than 0 mV vs. normal hydrogen electrode (NHE) in aqueous solution at a biologically relevant pH (e.g., a pH of 6.5-7.5 or 7.2-7.4, including all 0.1 pH values and ranges therebetween). In various other examples, a compound exhibits a reduction potential (Eₒ) more negative than 100, 0, −150, −200, −300, −400, −500, or −600 mV vs. normal hydrogen electrode (NHE) in aqueous solution at a biologically relevant pH (e.g., a pH of 6.5-7.5 or 7.2-7.4, including all 0.1 pH values and ranges therebetween). In various other examples, a compound or compound of the present disclosure exhibits a reduction potential (Eₒ) 450 to −600 mV (e.g., less than 0 to −600 mV), including all 0.1 mV values and ranges therebetween, vs. normal hydrogen electrode (NHE) in aqueous solution at a biologically relevant pH (e.g., a pH of 6.5-7.5 or 7.2-7.4, including all 0.1 pH values and ranges therebetween).

The imaging methods may use magnetic resonance-based imaging methods. Non-limiting examples of such methods include, magnetic resonance imaging (MM). Without intending to be bound to any particular theory, it is considered that the iron-based MRI contrast agents described herein (e.g., as high-spin, trivalent Fe(III)) produce contrast by the same paramagnetic mechanism as Gd(III) agents and are in small molecule form as coordination complexes, i.e., they are not nanoparticles. The imaging methods may also use positron emission tomography (PET) methods. PET is a diagnostic technique that monitors a positron-emitting radiotracer. The ⁶⁸Ga isotope is increasingly being used for PET given its favorable properties as a radiotracer. For example, ⁶⁸Ga has a short half-life of 68 minutes and produces predominantly positron (β+) emission. It may be desirable that the compound comprises one or more positron-emitting gallium cation(s) (e.g., ⁶⁸Ga isotope cation(s). It is relatively straightforward to commercially produce ⁶⁸Ga. The imaging methods may use magnetic resonance based imaging methods and/or positron emission tomography methods.

It is desirable that the electronic relaxation time of the high-spin Fe(III) centers are sufficiently long (e.g., greater than 1×10⁻¹¹ s or 3×10⁻¹¹ s), so that it is not the limiting factor in the correlation time constant as expressed in equation 4 at field strengths of 1.5 Tesla or greater. This can be accomplished by, for example, using ligands that produce high symmetry at the Fe(III) center. It is desirable that the zero field splitting factor (D) is small given that (T₁ₑ)⁻¹ is directly proportional to D² for high-spin Fe(III) complexes in an axially distorted complex.

One or more compound(s) may be covalently bound and/or non-covalently bound to a protein, such as, for example, human serum albumin, which is the predominant protein in the blood, and/or meglumine. Without intending to be bound by any particular theory. This approach is considered to slow the rotational correlation time and increase the relaxivity of the tethered compound(s) (e.g., at field strengths of 3 Tesla) and to increase the residency time of the contrast agent in the blood.

The following Statements describe various examples of the compounds, ligands, compositions, and methods of the present disclosure:

Statement 1. A compound (which may be a self-assembled cage) of the present disclosure (e.g., comprising: at least two structural units (which may be referred to as ligands), each structural unit comprising at least one spacer group (which may be referred to a linker group) and two or more donor groups (e.g., 2, 3, 4, or the like) (which may be referred to as chelating groups and may have one or more (e.g., one or two) group(s) that coordinate to an iron(III) cation (e.g., a high-spin iron(III) cation)), a gallium(III) cation (e.g., a naturally-occurring stable gallium isotope or a radioactive gallium isotope (e.g., ⁶⁸Ga, which is a positron emitter)), or a combination thereof, and at least one (e.g., at least two) iron(III) cations (which may be high-spin iron(III) cations) and/or at least one (e.g., at least two) gallium(III) cations. Each structural unit may have the same structure or one or more of the structural units may have a different structure relative to the other structural unit(s)).
Statement 2. The compound according to Statement 1, where the individual structural units are chosen from the following:

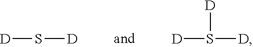

where S is spacer group and D is a donor group.
Statement 3. A compound according to Statement 1 or Statement 2, where each individual spacer group is a spacer group of the present disclosure (e.g., is chosen from

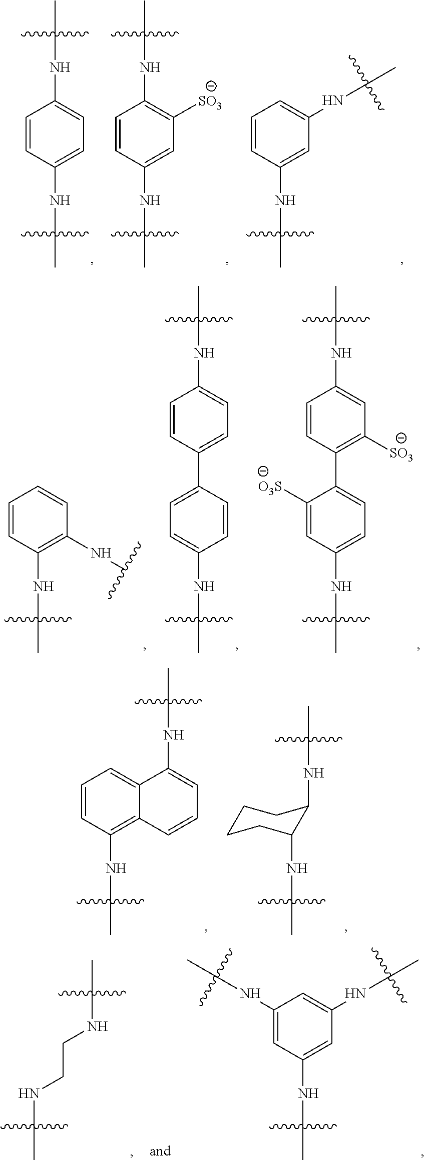

where an individual spacer group is covalently bound to at least one donor group via any open substitution site of the spacer group (e.g., via an amino group of the donor group). The spacer group (e.g., an aryl group of the spacer group) may be substituted with one or more (e.g., 1, 2, 3, etc.) sulfonic acid and/or sulfonic acid group(s)).
Statement 4. A compound according to any one of the preceding Statements, where each individual donor group is a donor group of the present disclosure (e.g., is chosen from

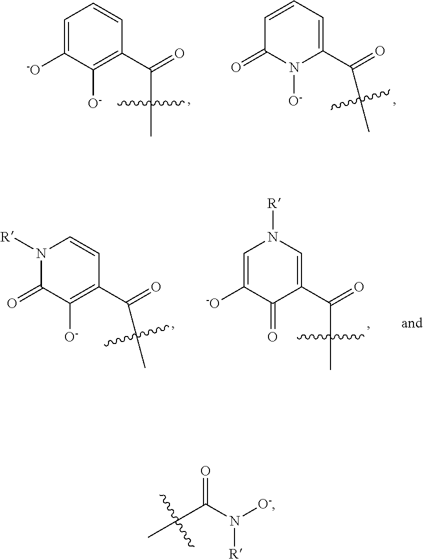

where R and R′ are independently chosen from alkyl groups (e.g., C₁ to C₁₂ alkyl groups (e.g., C₁, C₂, C₃, C₄, C₅, C₆, C₇, C₈, C₉, C₁₀, C₁₁, or C₁₂)), and where an individual donor group is covalently bound to a spacer group via any open substitution site of the donor group (e.g., via the carboxylic acid/carboxylate group of the donor group)).
Statement 5. A compound according to any one of the preceding Statements, where each individual structural unit is a structural unit of the present disclosure (e.g., is chosen from

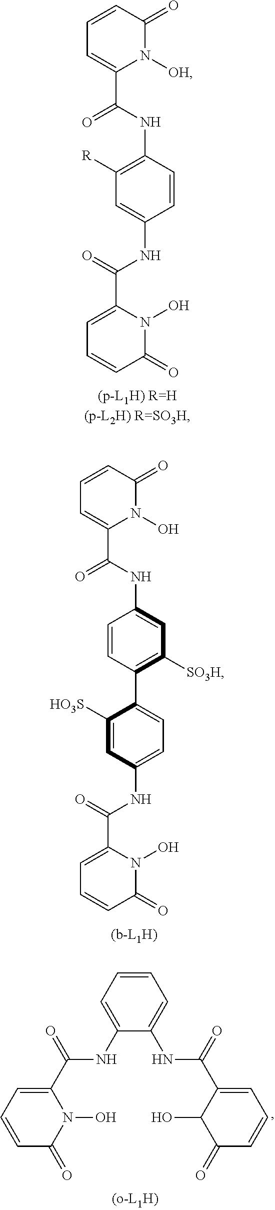

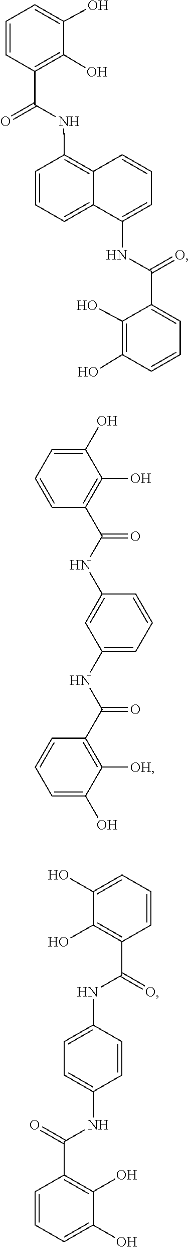

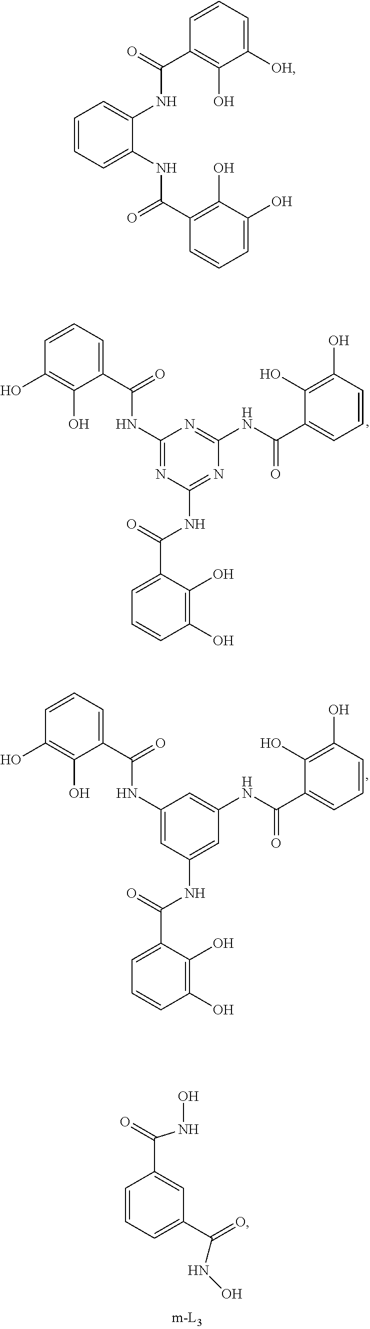

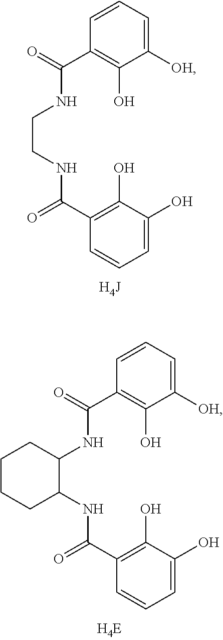

and the like, and aryl sulfonated analogs thereof, and at least partially or completely deprotonated (e.g., to provide one or more —O⁻ group(s)) analogs thereof (e.g., having two or more deprotonations), and combinations thereof).
Statement 6. A compound according to any one of the preceding Statements, where one or more of the individual iron(III) cation(s) has one or more ligand(s) (which may be a monodentate ligand, a bidentate ligand (e.g., a bridging ligand), or the like) that is not part of or a structural ligand.
Statement 7. A compound according to Statement 6, where the one or more ligand(s) that is not part of a structural ligand is, independently at each occurrence, chosen from (OR) groups (which may be bridging oxo groups), where R is independently at each occurrence, for example, alkyl groups (e.g., C₁-C₁₂ alkyl groups (e.g., C₁, C₂, C₃, C₄, C₅, C₆, C₇, C₈, C₉, C₁₀, C₁₁, or C₁₂)), carboxylate groups (e.g., C₁-C₁₂ carboxylate group), oxalate groups, carbonate groups, and the like, and combinations thereof.
Statement 8. A compound according to any one of the preceding Statements, where the compound has one of the following structures:
M₄L₆ (which may be an edge-directed tetrahedron structure), where the structural units may occupy the edges of the structure,
M₂L₃ (which may be a tris-helicate structure),
M₂L₂ (which may be a bis-helicate structure),
M₂L₂(OR)₂ (which may be a bis complex with two bridging alkoxide ligands), where the (OR) group is a bridging oxo group and R is, independently at each occurrence, a C₁-C₁₂ alkyl group (e.g., C₁, C₂, C₃, C₄, C₅, C₆, C₇, C₈, C₉, C₁₀, C₁₁, or C₁₂), which may comprise one or more fluorine substituent(s) (e.g., per-fluorinated, poly-fluorinated groups, such as, for example, a trifluoroethyl group and the like, and the like), or hydroxide group (—OH), M₄L₄ (which may be a face-directed tetrahedron structure), where the structural units may occupy the faces of the structure),
where M is an individual iron(III) cation (which may be a high-spin iron(III) cation) and L is an individual structural group (an individual ligand).
Statement 9. A compound according to Statement 8, where the spacer group and resulting compound structure are chosen from

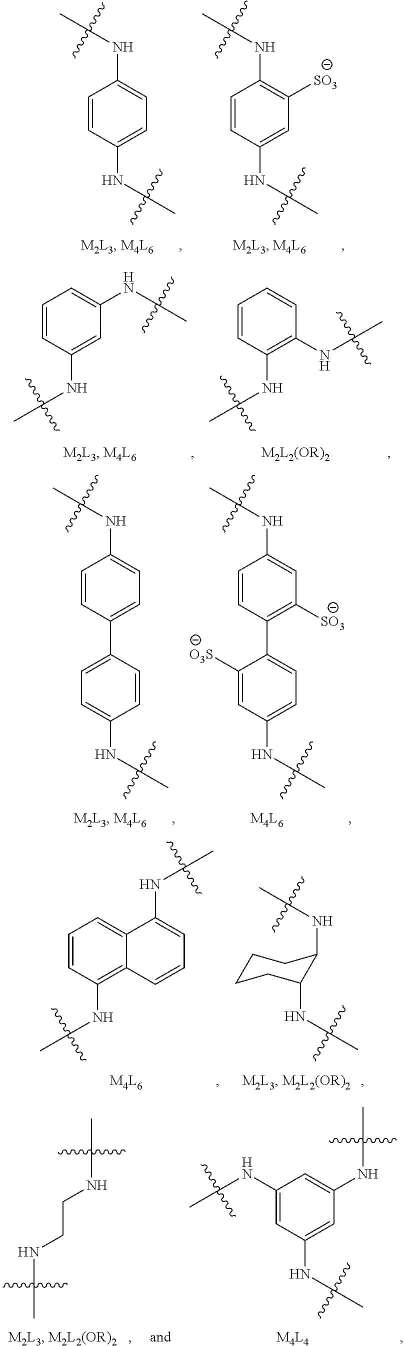

where each individual spacer group is covalently bound to at least one donor group via any open substitution site of the spacer group.
Statement 10. A compound according to any one of the preceding Statements, where the individual iron(III) cations are coordinatively saturated by the structural units and, optionally, non-structural unit ligand(s). E.g., the compound does not have or does have one or more coordinated water and/or hydroxide group(s).
Statement 11. A compound according to any one of the preceding Statements, where the compound does not exhibit detectible decomposition after 1 hour or more, 6 hours or more, 12 hours or more, or 24 hours or more at pH 7, at 37° C. in the presence of millimolar concentrations of carbonate and phosphate (e.g., 25 mM carbonate and 0.40 mM phosphate).
Statement 12. A compound according to any one of the preceding Statements, where the compound is paramagnetic or diamagnetic.
Statement 13. A compound according to any one of the preceding Statements, where the compound exhibits a r₁ relaxivity, which may per compound or per Fe(III) cation of a compound) of at least 1.5 mM⁻¹s⁻¹ at a field strength of 0.5, 1.5, 3 or 4.7 Tesla or greater.
Statement 14. A compound of any one of the preceding Statements, where the compound has the following structure

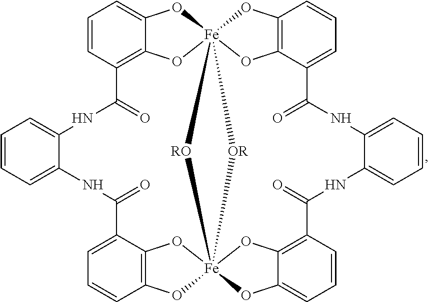

where R is an alkyl group or H group,

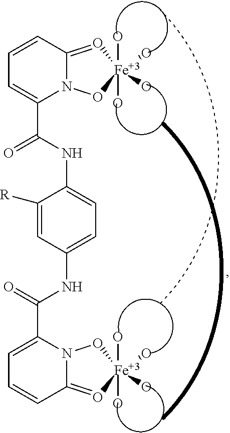

where the dotted and solid lines are coordinated ligands (which individually may be the specific ligand depicted),

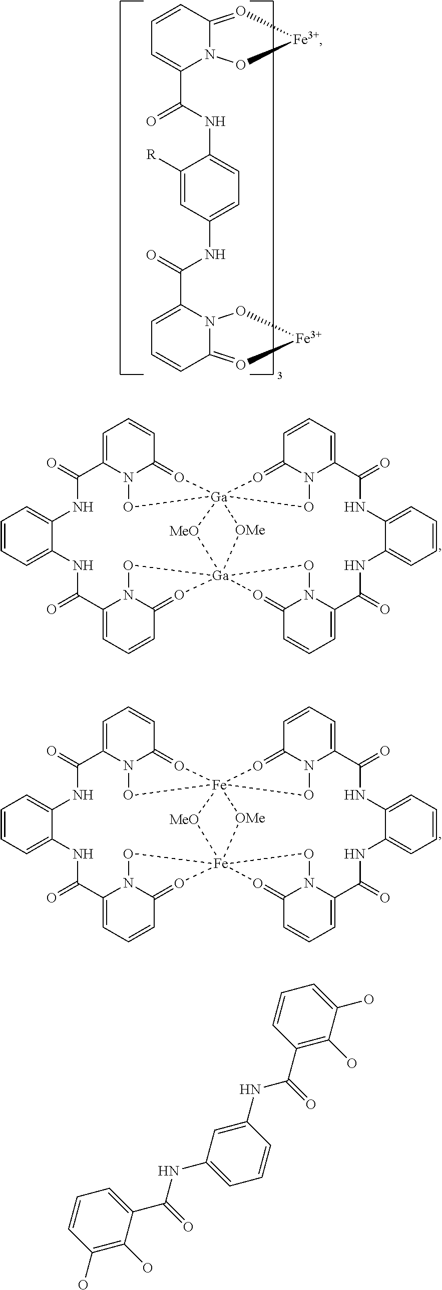

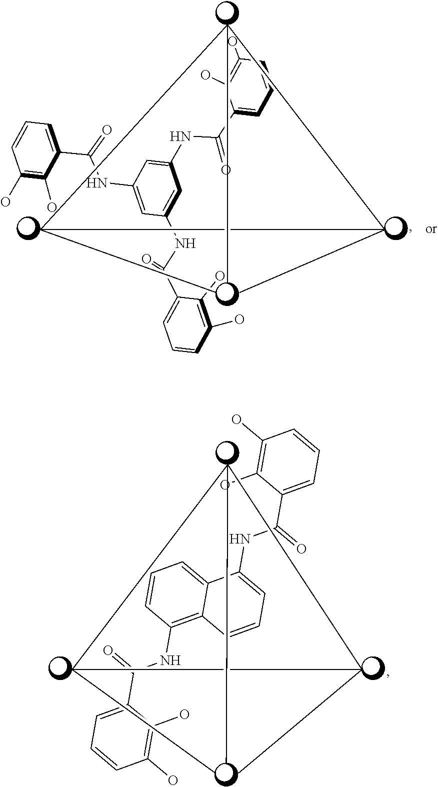

where the spheres at the vertices of the structure are independently chosen from iron(III) cations and gallium(III) cations, which individually may be high-spin iron(III) cations, or gallium(III) cations, which may individually be a naturally-occurring stable gallium isotope or a radioactive gallium isotope (e.g., ⁶⁸Ga, which is a positron emitter)).
Statement 15. A composition comprising one or more compound(s) of the present disclosure (e.g., one or more compound(s) of any one of Statements 1-14) and a pharmaceutically acceptable carrier.
Statement 16. A composition according to Statement 15, where the composition further comprises human serum albumin and/or meglumine, which may be covalently or non-covalently bound to the compound.
Statement 17. A method to obtain an image of at least a portion of a cell, organ, vasculature or tissue comprising: contacting the cell, organ, vasculature, or tissue with one or more compound(s) and/or one or more composition(s) of the present disclosure (e.g., one or more compound(s) of any one of Statements 1-14 and/or one or more composition(s) of any one of Statements 15-16), and imaging at least a portion of the cell, organ, vasculature, or tissue to obtain an image of the portion of a cell, organ, vasculature, or tissue, where the image may be obtained by using magnetic resonance and/or positron emission.
Statement 18. A method according to Statement 17, where the cell, organ, vasculature, or tissue is part of an individual.
Statement 19. A method according to Statement 17 or 18, where the image is obtained using magnetic resonance imaging (MM) and/or positron emission tomography (PET).
Statement 20. A method according to any one of Statements 17-19, where the compound(s) is/are a T₁ agent or T₁ agents and/or T₂ agent or T₂ agents.

The steps of the method described in the various examples disclosed herein are sufficient to carry out the methods of the present disclosure. Thus, in an example, a method consists essentially of a combination of the steps of the methods disclosed herein. In another example, a method consists of such steps.

The following examples are presented to illustrate the present disclosure. They are not intended to limiting in any matter.

### Example 1

This example provides a description of compounds of the present disclosure and method of making and characterization of the compounds.

2,3-Dimethoxy-Benzoyl Chloride. 2,3-dimethoxy-benzoic acid (3.00 g, 16.5 mmol) was added to 10 mL of dry dichloromethane under argon and stirring. The solution was cooled to 0° C. upon which thionyl chloride (1.44 mL, 19.8 mmol) was added drop wise. A catalytic amount of dimethylformamide was added shortly after. The solution was allowed to return to room temperature overnight. Volatiles were removed using rotary evaporation yielding a pale yellow wax solid (3.16 g, 15.8 mmols) 96% yield. The product 2,3-dimethoxy-benzoyl chloride was used without further purification.

1,5-Bis(2,3-dimethoxybenzamido)naphthalene (Me₄A). 2,3-dimethoxy-benzoyl chloride (2.00 g, 10.0 mmol) was added to round bottom with 1,5-diaminonapthalene (712 mg, 4.5 mmol). 15 mL of dichloromethane added to round bottom followed by triethylamine (4.0 mL, 30 mmol). Solution was stirred overnight. White precipitate was collected by vacuum filtration and washed with cold ethanol and water isolating pure Me₄A (1.8207 g, 3.75 mmol) 83.3% yield. ¹H NMR (300 MHz, CDCl₃) δ 10.74 (s, 2H), 8.56 (d, J=7.6 Hz, 2H), 7.99-7.81 (m, 4H), 7.60 (t, J=8.1 Hz, 2H), 7.30-7.23 (m, 2H), 7.15 (dd, J=8.2, 1.6 Hz, 2H), 4.10 (s, 6H), 3.98 (s, 6H).

1,5-Bis(2,3-dihydroxybenzamido)naphthalene (H₄A). 1,5-Bis(2,3-dimethoxybenzamido)naphthalene (1.20 g, 2.47 mmol) was added to 50 mL of dichloromethane. The solution was purged with argon for 20 minutes with stirring. The solution was cooled to −78° C. before adding 1M boron tribromide (10 mL, 10.0 mmol) dropwise. The solution was allowed to stir overnight returning to room temperature. 10 mL of distilled water was added to react excess boron tribromide and complete hydrolysis. Volatile organics were removed using rotary evaporation. The remaining sample was refluxed at 100° C. and allowed to precipitate. H₄A was isolated using vacuum filtration as white solid. ¹H NMR (300 MHz, DMSO-d₆) δ 11.89 (s, 1H), 10.89 (s, 1H), 9.50 (s, 1H), 7.89 (dd, J=17.7, 7.9 Hz, 4H), 7.66-7.57 (m, 4H), 7.02 (dd, J=7.8, 1.5 Hz, 2H), 6.82 (t, J=7.9 Hz, 2H).

1,3-Bis(2,3-dimethoxybenzamido)benzene (Me₄B). 2,3-dimethoxy-benzoic acid (1.00 g, 5.49 mmol) was added to 50 mL of dichloromethane. 1,1′-carbonyldiamidazole (0.89 g, 5.49 mmol) was added to the solution and stirred for 45 minutes. 1,3-benzenediamine (0.30 g, 2.74 mmol) was added to the solution and allowed to react for 1 week. The solution was dried using rotary evaporation and recrystallized in dichloromethane/hexane. The collected solid was washed with cold ethanol and water to remove excess imidazole.

1,3-Bis(2,3-dihydroxybenzamido)benzene (H₄B). 1,3-Bis(2,3-dimethoxybenamido)benzene (0.20 g, 0.46 mmol) was added to 10 mL of dichloromethane and stirred for 20 minutes under argon. 1M boron tribromide (3.62 mL, 3.62 mmol) was added dropwise to the solution which was allowed to stir overnight. 10 mL of water was added to the mixture to react excess boron before evaporating to dryness using rotary evaporation to remove residual volatile organics. The solid was re-dissolved in water and refluxed at 100° C. forming a white precipitate. Solid H₄B was isolated using vacuum filtration.

1,2-Bis(2,3-dimethoxybenzamido)benzene (Me₄C). 2,3-dimethoxy-benzoyl chloride (2.20 g, 11.0 mmol) was added to 30 mL of dichloromethane under argon. o-henylenediamine (592 mg, 5.48 mmol) was added to the flask followed by triethylamine (2.5 mL, 17.9 mmol) and a catalytic amount of dimethylformamide. The solution was allowed to stir for 5 hours. The solution was brought to dryness using rotary evaporation. The solid was re-dissolved in dichloromethane and precipitated with hexanes producing needle like crystals (1.5 g). ¹H NMR (400 MHz, CDCl₃) δ 9.92 (s, 2H), 8.25-7.54 (m, 4H), 7.54-6.90 (m, 6H), 3.87 (d, J=2.6 Hz, 12H). ES(+)MS m/z 437.1 [M+H]⁺, 459.3 [M+Na]⁺, 894.9 [2M+Na]⁺.

1,2-Bis(2,3-dihydroxybenzamido)benzene (H₄C). 1,2-Bis(2,3-dimethoxybenzamido)benzene (500 mg, 1.14 mmol) was added to 20 mL of dichloromethane under argon. The solution was cooled to 0° C. before adding 1M boron tribromide (10 mL, 10 mmol) dropwise. The solution was allowed to stir overnight returning to room temperature. 10 mL of distilled water was added to the solution to react excess boron tribromide and complete hydrolysis. Volatile organics were removed using rotary evaporation. The remaining sample was refluxed at 100° C. allowing a precipitate to form. H₄C was isolated using vacuum filtration as an off-white solid. ¹H NMR (500 MHz, CD₃OD) δ 7.72 (d, 2H), 7.40 (d, J=7.9 Hz, 2H), 7.35 (d, 2H), 6.99 (d, J=7.4 Hz, 2H), 6.79 (t, J=7.9 Hz, 2H). ES(−)MS m/z 379.3 [M−H]⁻

K₅(Et₄N)₇[Fe₄A₆]. Synthesis of K₅(Et₄N)₇[Fe₄A₆] was adapted from a previous literature procedure. Ligand H₄A (0.100 g, 0.232 mmol) was added to 30 mL of dry methanol under argon. 1 mL of 0.464 N potassium hydroxide (0.464 mmol) prepared in dry methanol was added to the mixture. After 10 minutes of stirring the ligand dissolved and 1 mL of 0.464 N tetraethylammonium chloride (0.464 mmol) prepared in dry methanol was added to the solution. Fe(acac)₃ (0.15 mmol) was added to the solution which rapidly turned deep red. The solution stirred overnight forming a deep red precipitate which was collected using vacuum filtration. Crystals suitable for x-ray diffraction were grown in three days by vapor diffusion of acetone into a water/methanol mixture containing the complex under argon atmosphere. μ_{eff} is 5.1±0.1 per iron center as measured by the Evans method.

K₅(Et₄N)₇[Ga₄A₆]. Synthesis of K₅(Et₄N)₇[Ga₄A₆] followed the previous procedure substituting Ga(acac)₃ for its Fe(III) analog. Upon addition of Ga(acac)₃ the solution rapidly turned yellow. Overnight a pale yellow precipitate formed and was collected by vacuum filtration as K₅(Et₄N)₇[Ga₄A₆]. ¹H NMR (500 MHz, D₂O) δ 8.02 (d, J=7.4 Hz, 12H), 7.77 (d, J=8.2 Hz, 12H), 7.19 (d, J=6.8 Hz, 12H), 7.06 (t, J=8.0 Hz, 12H), 6.62 (d, =5.8 Hz, 12H), 6.49 (t, 12H).

K₄[Ga₂C₂]. Ligand H₄C (0.050 g, 0.132 mmol) was added to 20 mL of methanol under argon. K₂CO₃ (0.066 mmol) and Ga(acac)₃ (0.132 mmol) were added to the solution which rapidly turned yellow. The solution stirred overnight forming a pale yellow precipitate which was collected using vacuum filtration and washed with methanol. ¹H NMR (300 MHz, D₂O) δ 7.55 (dd, J=5.9, 3.5 Hz, 4H), 7.26 (dd, J=5.9, 3.6 Hz, 4H), 6.98 (dd, J=8.2, 1.6 Hz, 4), 6.45 (dd, J=7.4, 1.6 Hz, 4H), 6.33 (t, J=7.8 Hz, 4H).

K₄[Fe₂C₂]. Synthesis of K₄[Fe₂C₂] followed the previous procedure substituting Fe(acac)₃ for its Ga(III) analog. Upon addition of Fe(acac)₃ the solution rapidly turned deep red. Overnight a deep red precipitate formed and was collected by vacuum filtration and washed with methanol.

### Example 2

This example provides a description of the characterization and use of compounds of the present disclosure as contrast agents in imaging methods.

FIGS. 9-11 show magnetic resonance images of mice obtained using compounds of the present disclosure.

In vivo imaging in mice as shown in FIGS. 9-11. Efficacy of the Fe(III) complexes for in vivo contrast enhancement were studied on at 4.7 T Bruker preclinical MRI in a mouse model (BABC/cJ, Jackson Laboratory). Sealed phantoms were included for imaging sessions for signal normalization. Prior to administration of contrast agents, scans were acquired to serve as baseline values of enhancement. Two scan protocols were employed including: (1) a T₁-weighted, 3D, spoiled-gradient echo scan covering the mouse from thorax to tail to determine signal enhancement and (2) inversion-recovery, steady state free precession scans (IR-SSFP) to measure T₁ rates in the blood (inferior vena cava), kidneys, liver, gall bladder and back muscle. Compounds were injected intravenously via tail vein at a dose of 12.5, 25 or 50 μmol [Fe4A6]/kg and MR data was acquired continuously for up to 1 hour after injection to study distribution and clearance kinetics. Additional scans were acquired at 3 and 6 and 12 hours post-injection to characterize slower clearance rates by the biliary system.

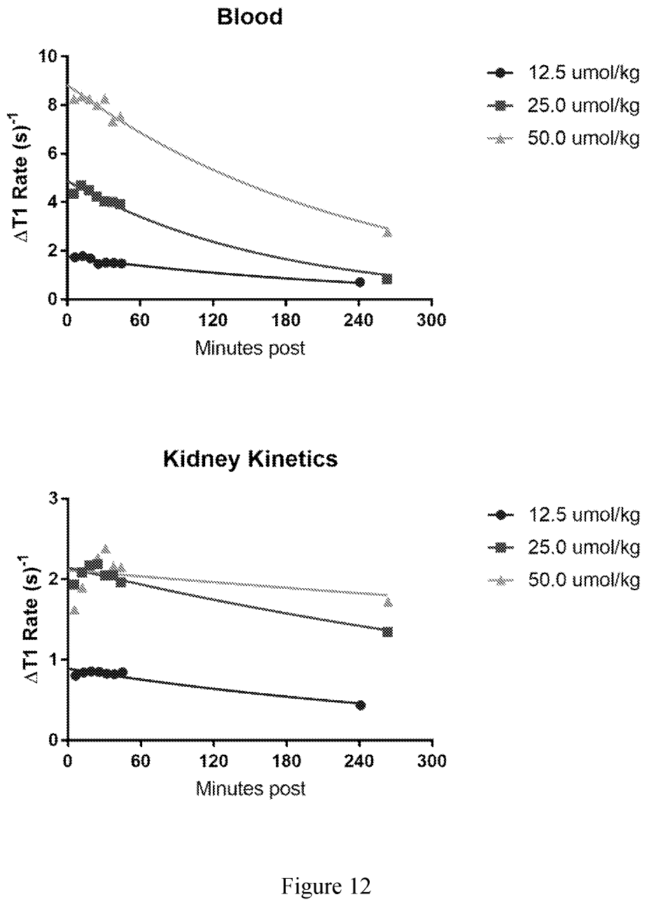

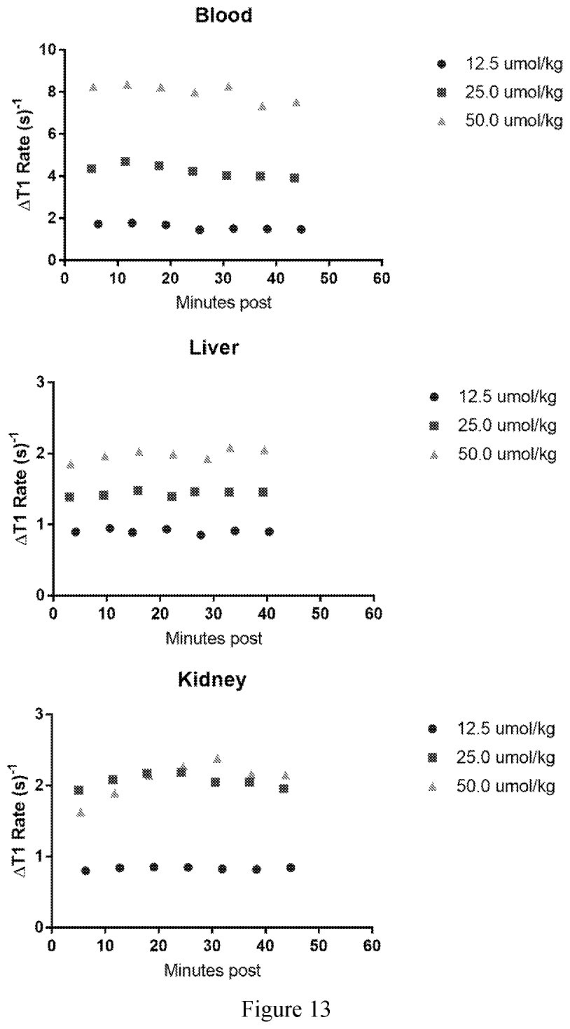

For SPGR datasets, signal intensities were normalized to the phantoms and signal increase for each organ was measured, as well as increase in contrast-to-noise ratios as compared to back muscle. This data is plotted in FIGS. 12-13 and shown in Table 1.

TABLE 1
Estimated plasma and renal half-life extrapolated from the plots of
ΔT₁ rate (s)⁻¹ over elapsed time (minutes) (Pharmacokinetics).
Plasma half-life is estimated to 2.5 hours independent of
dosage while renal half-life increases with dosage suggesting
saturation of the clearance mechanisms at higher dosages.

| Dosage (μmol/kg) | Location | Half-life (minutes) |
|---|---|---|
| 50 | Plasma | 164 |
| 25 | Plasma | 115 |
| 12.5 | Plasma | 174 |
| 50 | Kidney | 1153 |
| 25 | Kidney | 405 |
| 12.5 | Kidney | 251 |

TABLE 2
Calculated values of r₁ and r₂ for Fe(III) MOPs normalized to
complex concentration (r) and Fe(III) concentration
(r′) at 37° C., pH 7.2, 4.7 T.

| Complex | r₁ | r₂ | r_{1′} | r_{2′} |
|---|---|---|---|---|
| Fe₄A₆ | 6.5 | 12 | 1.6 | 3 |
| Fe₄A₆-HSA | 14 | 27 | 3.5 | 6.8 |
| Fe₂C₂(μ-OCH₃)₂ | 4.0 | 4.6 | 2.0 | 2.3 |
| Fe₂C₂(μ-OCH₃)₂-HSA | 9.3 | 14 | 4.7 | 6.8 |

Phantoms. (Table 2) Samples with variable concentrations (100, 200 and 400 μM) of Fe(III) complexes in 1×PBS solution with or without 35 g/L HSA at pH 7.4. T₁/T₂ relaxivity values were determined on a 4.7 Tesla MM system. Briefly, T₁ relaxation rates of serial dilutions were measured using an inversion-recovery, balanced steady-state free precession (bSSFP) acquisition with the following parameters: TE/TR=1.5/3.0 ms, flip angle=30°, inv. repetition time=10 s, segments=8, frames=100. T₂ relaxation rates were measured using a multi-echo, Carr-Purcell-Meiboom-Gill (CPMG) sequence with a fixed TR of 4200 ms and TE times ranging from 20-1200 ms in 20 ms increments. The relaxation rate of each sample was calculated using non-linear regression analysis within MATLAB (MathWorks, Natick Mass.) and relaxivities were then calculated by linear regression (concentration vs. relaxation rate).

FIGS. 12-13 show pharmacokinetic data for compounds of the present disclosure. Compounds were injected intravenously via tail vein at a dose of 12.5, 25 or 50 μmol [Fe]/kg and MR data was acquired continuously for up to 1 hour after injection to study distribution and clearance kinetics. Additional scans were acquired at 4 hours post-injection to characterize slower clearance rates by the biliary system.

For SPGR datasets, signal intensities were normalized to the phantoms and signal increase for each organ was measured, as well as increase in contrast-to-noise ratios as compared to back muscle.

### Example 3

This example provides a description of the characterization of compounds of the present disclosure.

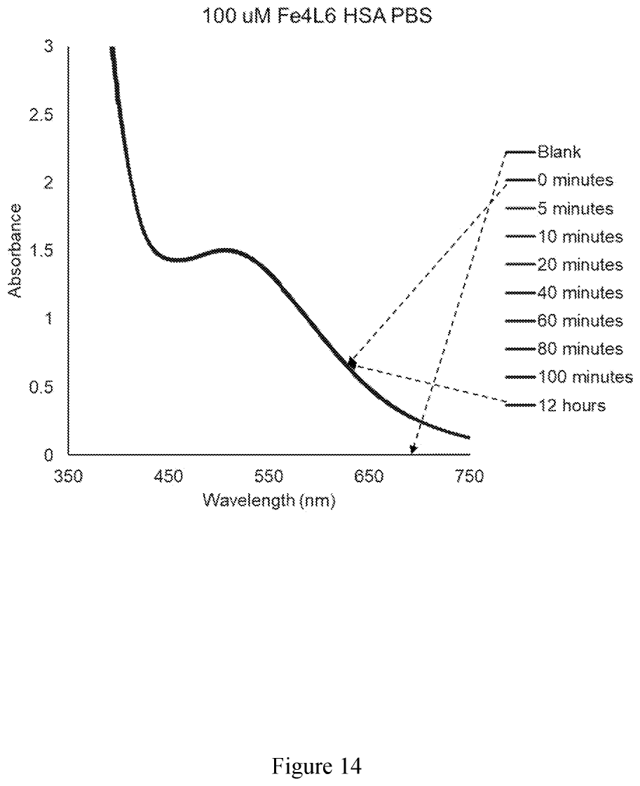

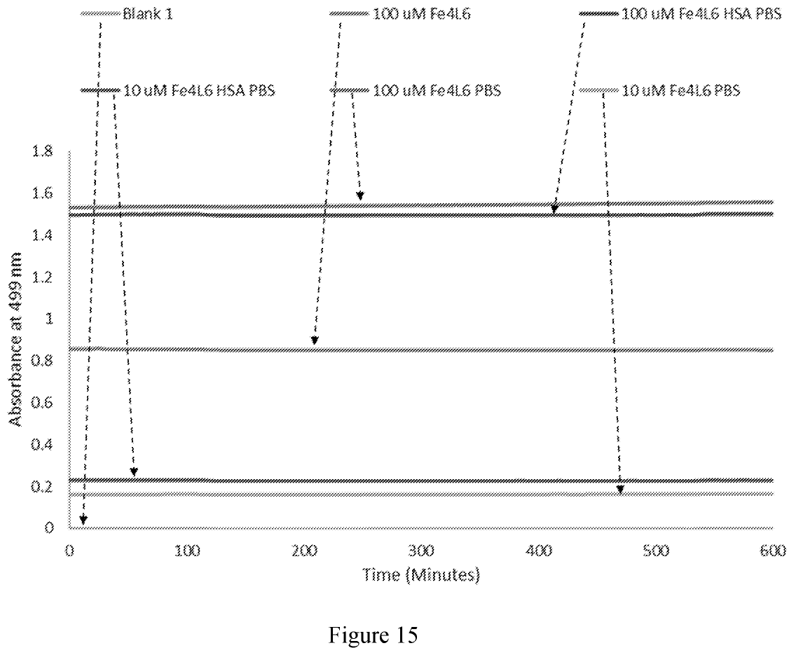

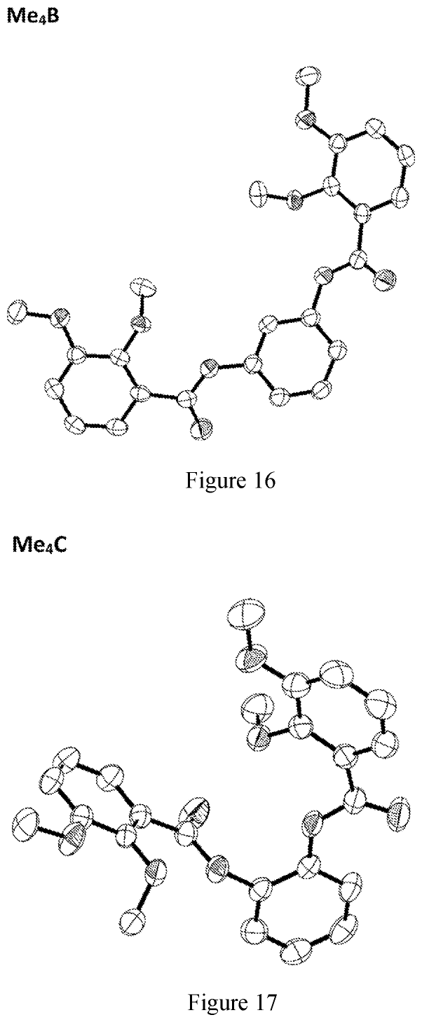

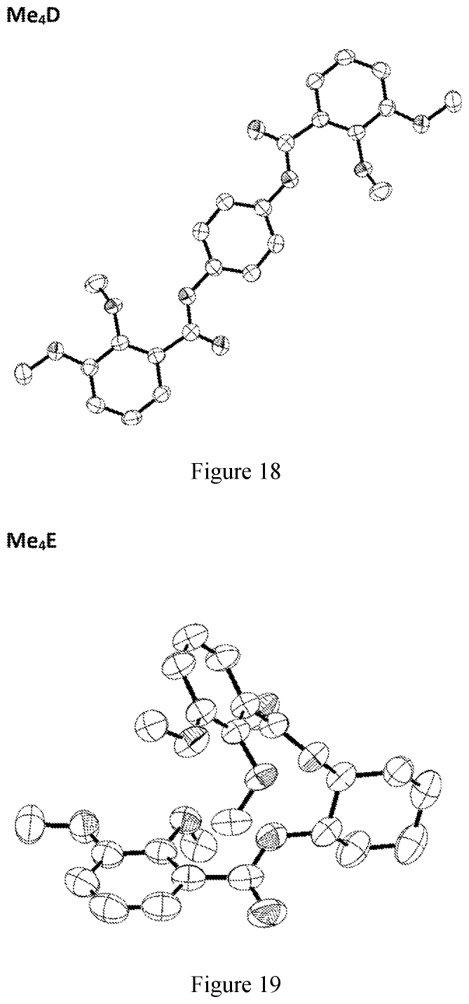

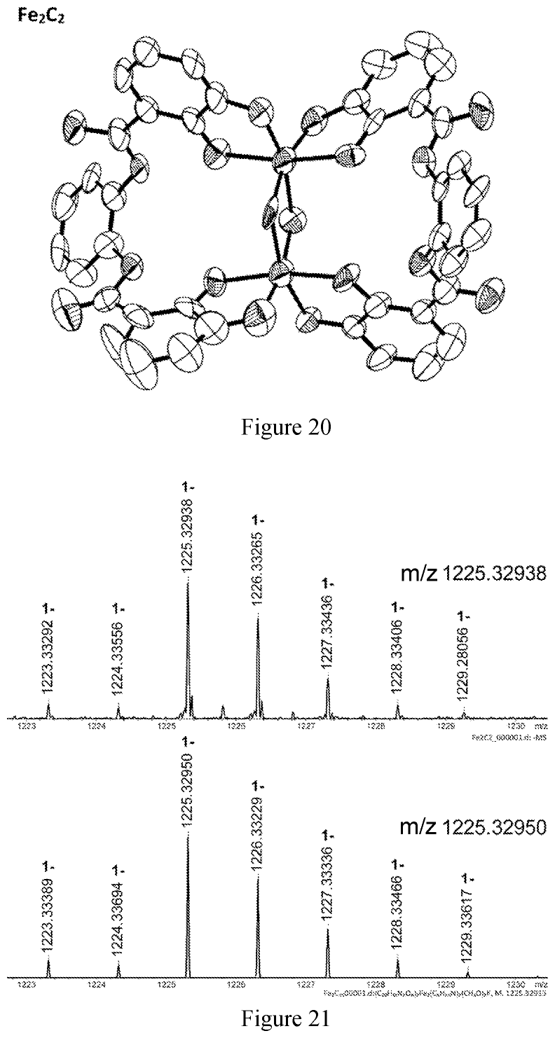

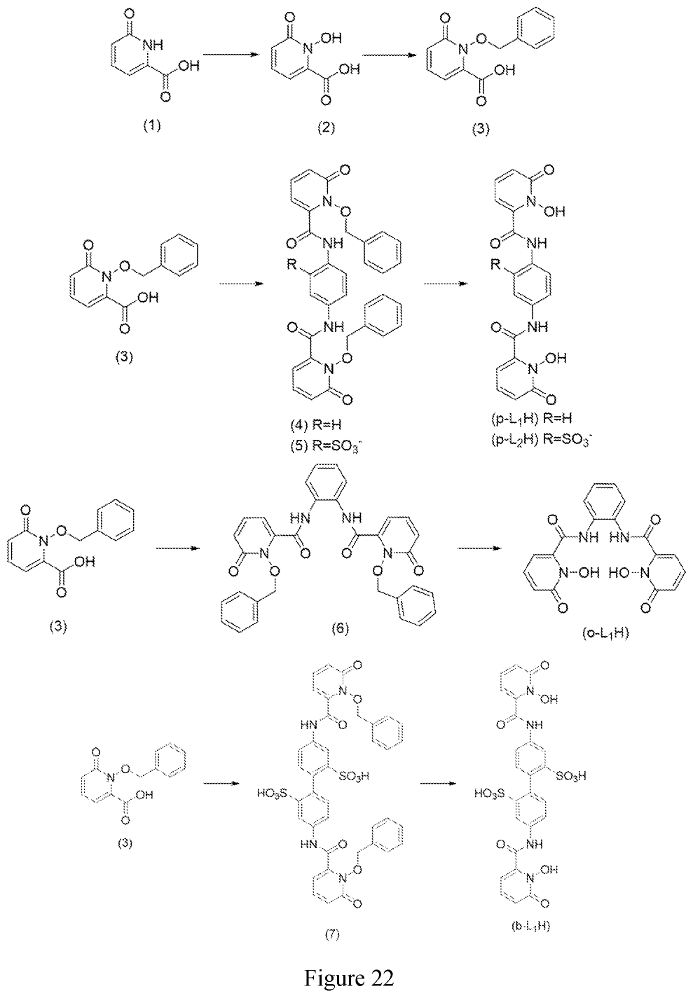

FIGS. 14 and 15 show UV-VIS data for a compound of the present disclosure. K₅(NEt₄)₇[Fe₄A₆] complex was dissolved in phosphate buffered saline solution to give concentrations of 10 or 100 μM. HSA concentrations were 0.6 mM. Solutions were added to cuvettes and placed in a Beckman spectrophotometer equipped with temperature probe and kinetics program. The absence of any change in the spectrum of the complex over time attests to the stability or inertness of the complex under these biologically relevant conditions.

### Example 4

This example provides a description of the characterization of compounds of the present disclosure.

H₄E—Under inert conditions, trans-1,2-bis(2,3-dimethoxybenzamido)cyclohexane was dissolved in dichloromethane (˜0.5 mmol per 15.0 mL). A 30-fold excess of boron tribromide per methoxy group was carefully added into the reaction vessel, immediately forming an orange solution, which was left stirring overnight. Excess boron tribromide was quenched via the careful addition of water, forming a red precipitate. The mixture was refluxed overnight, yielding a white precipitate that was isolated by filtration and washed with water. The product was obtained in a 57% yield. ¹³C NMR (75 MHz, dmso) δ 169.80 (s), 149.85 (s), 146.48 (s), 119.17 (s), 118.21 (s), 117.74 (s), 115.47 (s), 52.46 (s), 40.65 (d, J=20.9 Hz), 40.47-40.45 (m), 40.23 (s), 39.95 (s), 39.54 (d, J=21.0 Hz), 39.12 (s), 31.88 (s).

TABLE 3
FT-IRCMS fragment assignment for Fe₂C₂.

| Fragments | MxLy | Charge | m/z |
|---|---|---|---|
| [FeC(MeO)] | M1L1 | 1− | 463.025 |
| [Fe₂C₂] | M2L2 | 2− | 432.007 |
| (NEt₄)₂[Fe₂C₂(μ-MeO)₂] | M2L2 | 2− | 593.185 |
| K(NEt₄)₂[Fe₂C₂(μ-MeO)₂] | M2L2 | 1− | 1225.33 |

Ligand syntheses—The syntheses of scheme 1 were adapted from a previously reported procedure. Dichloromethane (DCM), tetrahydrofuran (THF) and triethylamine (TEA) were distilled and stored under argon over type 4A molecular sieves.

6-carboxy-1-hydroxy-2(1H)-pyridinone (6-carboxy-1,2HOPO) (2)—6-hydroxypicolinic acid (10.0100 g, 71.9574 mmol) was dissolved in 55 mL of trifluoroacetic acid and 40 mL of acetic acid under argon. 32% peracetic acid in dilute acetic acid (15.1 mL, 71.8 mmol) was slowly poured in and mixed under argon for 1 hour. The solution was moved to an oil bath at 80° C. overnight. The brown mixture was allowed to cool to room temperature after 16 hours of mixing. The flask was moved to the freezer for 2 hours to help precipitation. The precipitate was filtered out and washed with cold methanol. The white powder (6-carboxy-1,2 HOPO) was dried under vacuum for 5 hours and stored under argon. 6.9699 g (44.9352 mmol) of 6-carboxy-1,2HOPO was collected (62.6% yield). ESI-MS: m/z 154.2 [M−H⁺]⁻. ¹H NMR (500 MHz, DMSO-d₆): ¹H NMR (500 MHz, DMSO-d₆): δ 6.65 (m, 2H, ArH), 7.45 (t, 2H, ArH).

1-benzyloxy-6-carboxy-2(1H)-pyridinone (1,2HOPOBn) (3)—6-carboxy-1,2 HOPO (6.9699 g, 44.9352 mmol) and potassium carbonate (12.4246 g, 89.8965 mmol) were added to a round bottom flask under argon. 190 mL of methanol and benzyl bromide (6.9 mL, 58.1 mmol) were added. The mixture was refluxed under argon at 67° C. and became blue. The mixture continued stirring overnight and became a brown color. Excess potassium carbonate was filtered out and the solvent was reduced. The resulting brown and white solid was dissolved in distilled water. Concentrated hydrochloric acid was slowly added to the solution to form a precipitate. The precipitate was collected and washed with dilute hydrochloric acid and then distilled water. Some of the white powder was lost during the transfer. The powder was dried under vacuum over the course of two days. 6.7 g (27.3 mmol) of 1,2HOPOBn was collected (60.8% yield). ESI-MS: m/z 246.0 [M+H⁺]⁺. ¹H NMR (300 MHz, DMSO-d₆): ¹H NMR (300 MHz, DMSO-d₆): δ 5.26 (s, 2H, OCH), 6.52 (d, 2H, ArH), 6.71 (d, 2H, ArH), 7.39-7.50 (m, 6H, ArH).

N,N′-(1,4-phenylene)bis(1-(benzyloxy)-6-oxo-1,6-dihydropyridine-2-carboxamide) (1,2HOPOBn-pPDA) (4) —1,2HOPOBn (0.6590 g, 2.687 mmol) was added to a flask under argon. 1 drop of dimethylformamide (DMF) was added and an excess of 5 mL of thionyl chloride was added. The white mixture was mixed overnight under argon. The mixture became a yellow solution overnight. The remaining thionyl chloride was removed under vacuum. The flask was moved to a 0° C. bath and 15 mL of THF and 1 mL of TEA was added. p-phenylenediamine (0.1180 g, 1.091 mmol) was added to the solution as a powder. The solution was allowed to warm to room temperature over the course of 2 hours with stirring. A beige powder (triethylammonium chloride) was filtered out of the solution which lost color and became whiter as it was washed with THF. The filtrate was collected and the solvent was removed to give a dark brown oil. The oil was dissolved in 50 mL DCM and extracted with 30 mL of 0.1 M aqueous sodium bicarbonate. The NaHCO₃ layer was extracted twice with 20 mL DCM. The organic layers were combined and dried over MgSO₄. The magnesium sulfate was filtered out and the filtrate was dried to a brown powder. (0.3436 g, 0.6114 mmol) in a 52% yield. ESI-MS: m/z 563.1 [M+H⁺]⁺, 585.1 [M+Na⁺]⁺. ¹H NMR (300 MHz, DMSO-d₆): δ 5.29 (s, 4H, OCH), 6.51 (d, 2H, ArH), 6.71 (d, 2H, ArH), 7.36 (m, 10H, ArH), 7.54 (t, 2H, ArH), 7.68 (s, 4H, ArH), 10.93 (s, 2H, ArNH).

N,N′-(1,4-phenylene)bis(1-hydroxy-6-oxo-1,6-dihydropyridine-2-carboxamide) (1,2HOPO-pPDA) (p-L₁H) —1,2HOPOBn-pPDA (300 mg) was added to a flask under argon. The powder was dissolved in 30 mL DCM in a −78° C. bath. 1 M BCl₃ in DCM (14 mL, 14 mmol) was added through a glass syringe and metal needle. The reaction was allowed to stir overnight and to come to room temperature. The organic solvents were removed under vacuum and the brown oil was quenched with water and heated to 90° C. for 30 minutes. A brown precipitate was collected and washed with ether and dried under vacuum in 14% yield. ESI-MS: m/z 381.2 [M−H⁺]⁻. ¹H NMR (300 MHz, DMSO-d₆): δ 6.41 (d, 2H, ArH), 6.61 (d, 2H, ArH), 7.42 (t, 2H, ArH), 7.65 (s, 4H, ArH), 10.84 (s, 2H, ArNH).

2,5-bis(1-(benzyloxy)-6-oxo-1,6-dihydropyridine-2-carboxamido)benzenesulfonic acid (1,2HOPOBn-pPDASO₃) (5) —1,2 HOPOBn (0.2412 g, 0.9835 mmol) was added to a round bottom flask under argon. A drop of DMF and 3 mL of thionyl chloride was added through a syringe and the powder was dissolved under reflux at 78° C. In a separate flask 1,4-phenylenediamine 2-sulfonic acid (0.0983 g, 0.5223 mmol) was dissolved in 40 mL DCM and 1 mL TEA. After 3 hours of mixing excess thionyl chloride was removed from the acyl chloride solution. The 1,4-phenylenediamine 2-sulfonic acid solution and the acyl solution were combined. White smoke arose and dissipated. The solution was moved to an oil bath at 40° C. under argon and stirred for 16 hours. The solution was brown and clear. The solvent was removed to get an orange oily solid. The solid was dissolved in 1% methanol in DCM and separated on a silica gel flash column with a mobile phase of 1% methanol in DCM with a gradient by increasing the percentage of methanol up to 20%. Evaporation of solvent gave a yellow-orange solid. ESI-MS: m/z 641.6 [M−H⁺]⁻.

2,5-bis(1-hydroxy-6-oxo-1,6-dihydropyridine-2-carboxamido)benzenesulfonic acid (1,2HOPO-pPDASO₃) (p-L₂H)—The deprotection of this compound was carried out by using a similar procedure to deprotection of (5). ESI-MS: m/z 461.3 [M−H⁺]⁻.

N,N′-(1,2-phenylene)bis(1-(benzyloxy)-6-oxo-1,6-dihydropyridine-2-carboxamide) (1,2HOPOBn-oPDA) (6)—1,2HOPOBn (0.95 g, 3.9 mmol) was placed in a round bottom flask under argon. One drop of DMF and 8 mL of thionyl chloride was added. The white mixture was mixed overnight under argon and became a yellow solution. The excess thionyl chloride was removed under vacuum. The acid chloride was dissolved in 35 mL DCM and placed in an addition flask. o-Phenylenediamine (0.14 g, 1.3 mmol) was added to a different flask. 20 mL DCM and 5 mL of 30% potassium carbonate in distilled water were added to the flask with o-phenylenediamine. With vigorous stirring the acid chloride solution was dropped in slowly over the course of an hour under argon. The mixture was bright yellow. After the acid chloride was added, the mixture was allowed to stir overnight. The brown and yellow mixture was transferred to a separatory funnel where the organic layer was collected and the solvent removed. The crude brown oil was dissolved in 2% methanol in DCM and eluted on a silica column with the same solvent. The yellow band was collected first and the solvent removed to give a pale brown powder (0.6192 g, 1.101 mmol) in 85% yield. ESI-MS: m/z 563.3 [M+H⁺]⁺. ¹H NMR (400 MHz, CDCl₃): δ 5.21 (s, OCH, 4H), 6.64 (d, ArH, 2H), 6.71 (d, ArH, 2H), 7.10-7.50 (m, ArH, CHCl₃), 8.61 (s, NH, 2H).

N,N′-(1,2-phenylene)bis(1-hydroxy-6-oxo-1,6-dihydropyridine-2-carboxamide) (1,2HOPO-oPDA) (o-L₁H)—1,2HOPOBn-oPDA (0.6192 g, 1.101 mmol) was dissolved in 24 mL 1:1 hydrochloric acid and acetic acid. The solution was a faint red and yellow color. The solution stirred under argon for 64 hours. The solvent was removed to give a pale brown powder that was washed with ether twice and dried under vacuum. 0.35 g (0.92 mmol) of o-L₁H was collected (83% yield). ESI-MS: m/z 381.4 [M−H⁻]⁻, 763.3 [2M−H⁺]⁻, 801.3 [2M-2H⁺+K⁺]⁻. ¹H NMR (300 MHz, DMSO-d₆): δ 6.66 (m, ArH, 4H), 7.25-7.50 (m, ArH, 4H), 7.69 (t, ArH, 2H), 10.51 (s, NH, 2H).

4,4′-bis(1-(benzyloxy)-6-oxo-1,6-dihydropyridine-2-carboxamido)-[1,1′-biphenyl]-2,2′-disulfonic acid (1,2HOPOBn-BDA) (7)—1,2HOPOBn was placed in a round bottom flask under argon. One drop of DMF and 5 mL of thionyl chloride was added. The white mixture was mixed overnight under argon and became a yellow solution. The excess thionyl chloride was removed under vacuum. The acid chloride was dissolved in 35 mL DCM and placed in an addition flask. Benzidine-2,2′-disulfonic acid was dissolved in 50 mL 20% sodium carbonate in distilled water in a separate flask. The acid chloride was dropped in to the water solution with vigorous stirring over the course of two hours. The mixture was allowed to stir overnight. The mixture was placed in a separatory funnel and the water layer was collected and the solvent removed. Methanol was added to the white powder and insoluble material was filtered out. The filtrate solvent was removed and 20% methanol in dichloromethane was added to the white powder. Insoluble material was filtered out and the filtrate was eluted on a silica column. A gradient of 20% methanol in dichloromethane to 100% methanol was used as the eluent. The solvent in the first band was reduced to give a white powder. ESI-MS: m/z 797.1 [M−H⁻]⁻.

Ga₂(o-L)₂(OMe)₂:

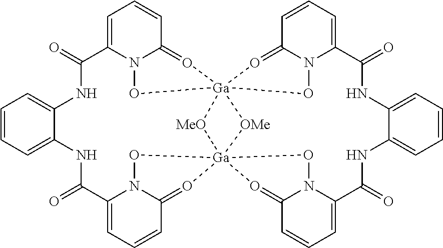

The ligand, o-L₁H (20.7 mg, 0.054 mmol), was dissolved in 2 mL methanol. Gallium acetylacetonate (21.0 mg, 0.057 mmol) was dissolved in 3 mL methanol and two drops of pyridine was added. The two solutions were added together. A white precipitate formed immediately. The mixture was refluxed for two hours and cooled to room temperature. The white precipitate was filtered out and washed with methanol.

Fe₂(o-L)₂(OMe)₂: The ligand, o-L₁H (73.5 mg, 0.192 mmol) was dissolved in 6 mL methanol. Iron tris(aceylacetonate) (71.6 mg, 0.203 mmol) was dissolved in 9 mL methanol and six drops of pyridine was added. The two solutions were added together. An orange precipitate formed immediately. The mixture was refluxed for two hours and cooled to room temperature. The orange precipitate was filtered out and washed with methanol. ESI-MS: m/z 957.1 [M+Na⁺].

Although the present disclosure has been described with respect to one or more particular example(s), it will be understood that other example(s) of the present disclosure may be made without departing from the scope of the present disclosure.

1. A compound comprising:
at least two structural units, each structural unit comprising at least one spacer group and two or more donor groups, which have one or more group(s) that coordinate to an iron(III) cation, a gallium(III) cation, or a combination thereof, and
at least two iron(III) cations, at least two gallium(III) cations, or a combination of one or more iron(III) cation(s) and one or more gallium(III) cation(s),
wherein each structural unit has the same structure or one or more of the structural unit(s) have a different structure relative to the other structural unit(s)),
or a salt, a partial salt, a hydrate, a polymorph, or a stereoisomer thereof, or a mixture thereof.

2. The compound of claim 1, wherein the individual structural units are chosen from the following: 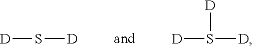
wherein S is spacer group and D is a donor group.

3. The compound of claim 1, wherein each individual spacer group is chosen from 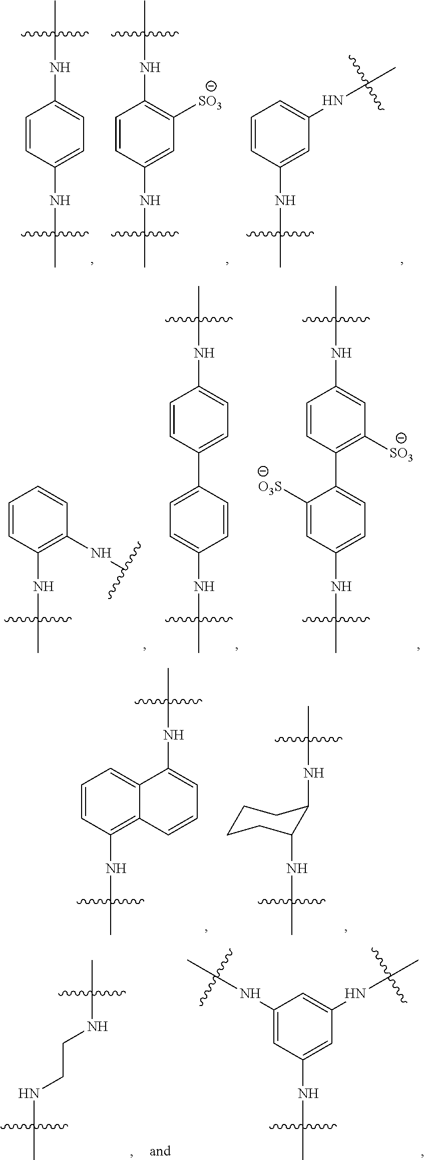
wherein each individual spacer group is covalently bound to at least one donor group via any open substitution site of the spacer group.

4. The compound of claim 1, wherein each individual donor group chosen from 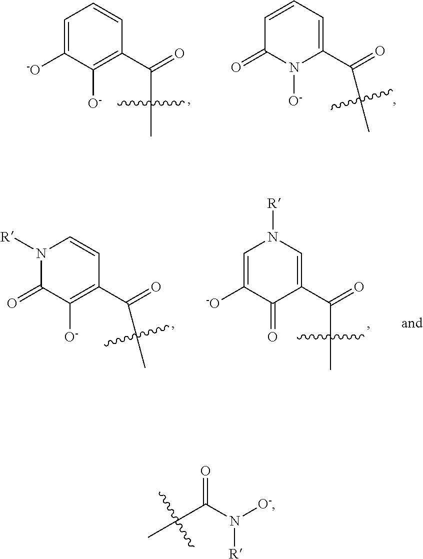
wherein R and R′ are independently chosen from alkyl groups,
wherein an individual donor group is covalently bound to a spacer group via any open substitution site of the donor group.

5. The compound of claim 1, wherein each individual structural unit is chosen from 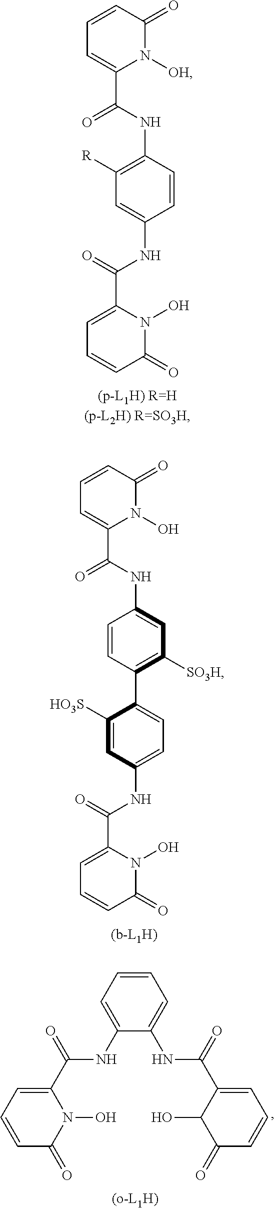 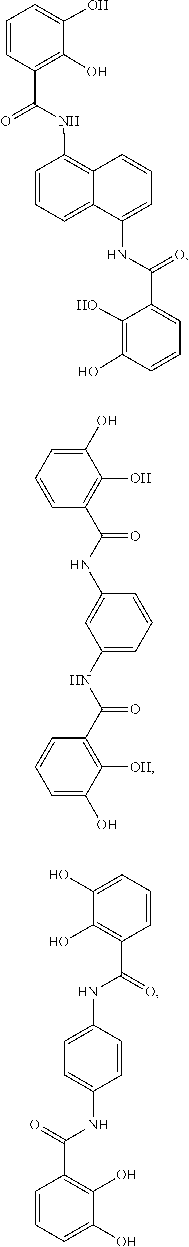 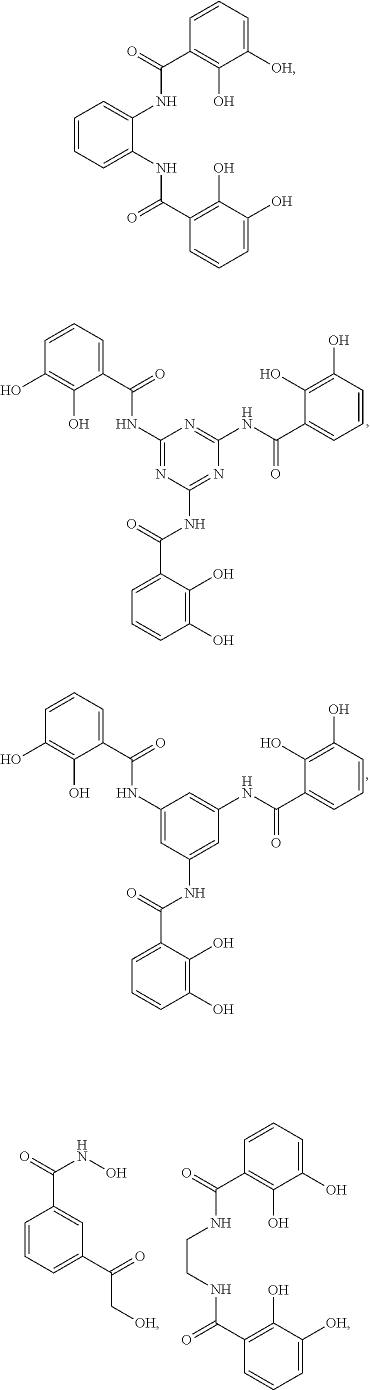 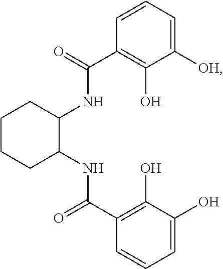
and aryl sulfonated analogs thereof, and at least partially deprotonated analogs thereof, and combinations thereof.

6. The compound of claim 1, wherein one or more of the individual iron(III) cation(s) has one or more ligand(s) that is/are not part of or a structural ligand.

7. The compound of claim 6, wherein the one or more ligand(s) that is/are not part of a structural ligand is/are, independently at each occurrence, chosen from (OR) groups, wherein R is independently at each occurrence chosen from alkyl groups, carboxylate groups, oxalate groups, carbonate groups, and combinations thereof.

8. The compound of claim 1, wherein the compound has one of the following structures:
an edge-directed tetrahedron structure (M₄L₆), wherein the structural units occupy the edges of the structure,
a tris-helicate structure (M₂L₃),
a bis-helicate structure (M₂L₂),
a bis complex with two bridging alkoxide ligands (M₂L₂(OR)₂), wherein the (OR) group is a bridging alkoxide group and R is independently at each occurrence an alkyl group, or hydroxide (OH), or
a face-directed tetrahedron structure (M₄L₄), wherein the structural units occupy the faces of the structure,
wherein M is an individual iron(III) cation and L is an individual structural group.

9. The compound of claim 8, wherein the spacer group and resulting compound structure are chosen from 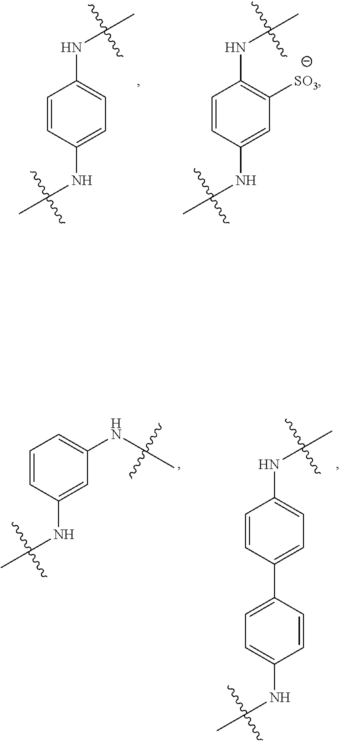
wherein the compound has the formula M₂L₃ or M₄L₆, 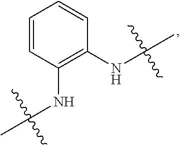 [SMILES: *Nc1ccccc1N*]
wherein the compound has the formula M₂L₂(OR)₂, wherein R is an alkyl group, 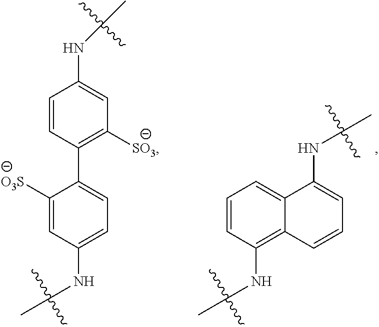
wherein the compound has the formula M₄L₆, 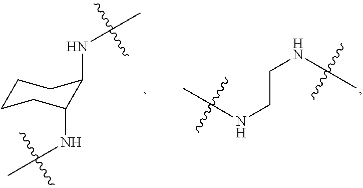 [SMILES: *NC1CCCCC1N* ; *NCCN*]
wherein the compound has the formula M₂L₃ or M₂L₂(OR)₂, wherein R is an alkyl group, and 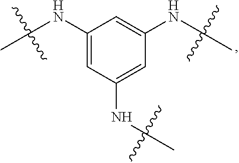 [SMILES: *Nc1cc(N*)cc(N*)c1]
wherein the compound has the formula M₄L₄,
wherein each individual spacer group is covalently bound to at least one donor group via any open substitution site of the spacer group.

10. The compound of claim 1, wherein the individual iron(III) cations are coordinatively saturated by the structural units and, optionally, non-structural unit ligand(s).

11. The compound of claim 1, wherein the compound does not exhibit detectible decomposition after 1 hour or more at pH 7, at 37° C., and in the presence of 25 mM carbonate and 0.40 mM phosphate.

12. The compound of claim 1, wherein the compound is paramagnetic or diamagnetic.

13. The compound of claim 1, wherein the compound exhibits a T₁ relaxivity of at least 1.5 mM⁻¹s⁻¹ at a field strength of 4.7 Tesla or greater.

14. The compound of claim 1, wherein the compound has the following structure 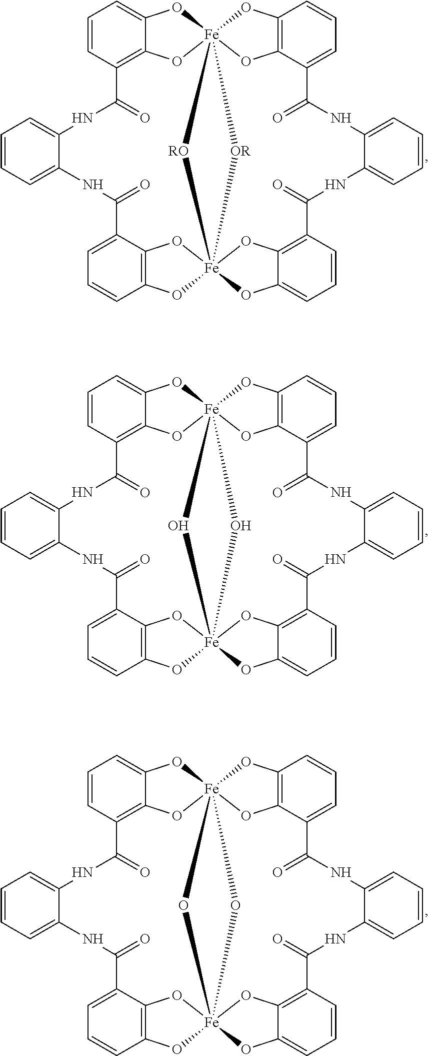
wherein R is, independently, at each occurrence, an alkyl group or H group, 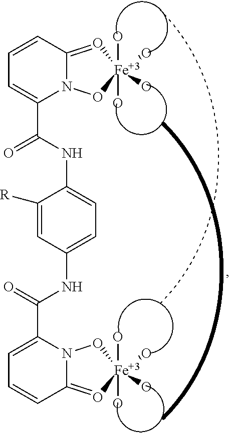
wherein the dotted and solid lines are coordinated ligands, 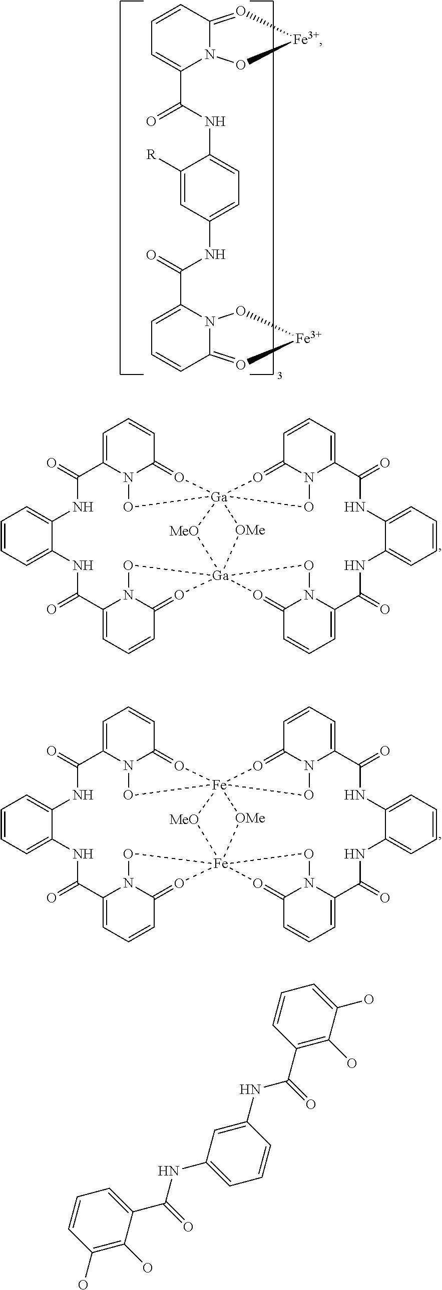 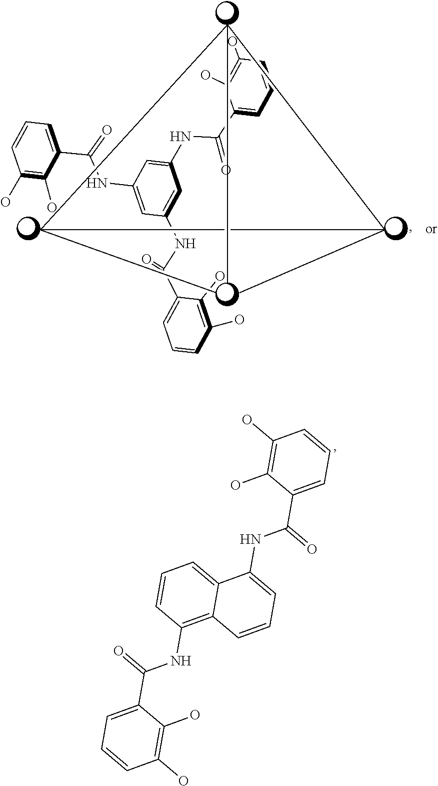
wherein the spheres at the vertices of the structure are independently chosen from iron(III) cations and gallium(III) cations, which individually may be high-spin iron(III) cations, or gallium(III) cations, which may individually be a naturally-occurring stable gallium isotope or a radioactive gallium isotope.

15. A composition comprising one or more compound(s) of claim 1 and a pharmaceutically acceptable carrier.

16. The compound of claim 15, wherein the composition further comprises human serum albumin and/or meglumine.

17. A method to obtain an image of at least a portion of a cell, organ, vasculature, or tissue comprising:
contacting the cell, organ, vasculature, or tissue with one or more compound(s) of claim 1, and
imaging at least a portion of the cell, organ, vasculature, or tissue to obtain an image of the portion of a cell, organ, vasculature, or tissue,
wherein the image is obtained by using magnetic resonance.

18. The method of claim 17, wherein the cell, organ, vasculature, or tissue is part of an individual.

19. The method of claim 17, wherein the image is obtained using magnetic resonance imaging (MRI) and/or positron emission tomography (PET).

20. The method of claim 17, wherein the compound(s) is/are a T₁ agent or T₁ agents and/or a T₂ agent or T₂ agents.
